SEQUENCE LISTING (1 sequence)

SEQ ID NO: 1 - DNA, 8441 bp, alpha proteobacterium C7
  5′UTR (1)..(44)
  misc_feature (45)..(7523): REP (nsP1-4)
  misc_feature (7501)..(7545): Sub-genomic Promoter
  misc_feature (7574)..(8293): SOI (i.e. GFP)
  3′UTR (8325)..(8441)
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggcgatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggcgcgcctaagtaactaactgactgagtgaatacagcagcaattggcaagctgcttacatagaactcgcggcgattggcatgccgccttaaaatttttattttatttttcttttcttttccgaatcggattttgtttttaatatttc

(19) United States
(12) Patent Application Publication
(10) Pub. No.: US 2022/0054611 A1
(43) Pub. Date: Feb. 24, 2022

(54) PATHOGEN-ASSOCIATED MOLECULAR PATTERN MOLECULES AND RNA IMMUNOGENIC COMPOSITIONS AND METHODS OF USING THE COMPOSITIONS FOR TREATING CANCER

(71) Applicant: IMMUNE DESIGN CORP., Rahway, NJ (US)

(72) Inventors: Peter Lars Aksel Berglund, Seattle, WA (US); Tinglan Tina Albershardt, Olympia, WA (US); Jan Henrik Ter Meulen, Mercer Island, WA (US); Jardin Alexandra Leleux, Seattle, WA (US)

(73) Assignee: IMMUNE DESIGN CORP., Rahway, NJ (US)

(21) Appl. No.: 17/413,853

(22) PCT Filed: Dec. 16, 2019
(86) PCT No.: PCT/US19/66444

§ 371 Date: Jun. 14, 2021

Related U.S. Application Data
(60) Provisional application No. 62/780,665, filed on Dec. 17, 2018.

(51) Int. Cl. A61K 39/00; A61K 39/39; A61K 45/06

(52) U.S. Cl. CPC A61K 39/0011; A61K 2039/55538; A61K 45/06; A61K 39/39

(57) ABSTRACT

The present invention provides novel compositions and methods for inhibiting tumor growth, treating cancer, and/or inducing immune responses. Such compositions and methods induce an immune response for the treatment of a variety of diseases including cancer.

### INCORPORATION BY REFERENCE OF MATERIAL SUBMITTED ELECTRONICALLY

Incorporated by reference in its entirety is a computer-readable amino acid/nucleotide sequence listing submitted concurrently herewith and identified as follows: Filename: 53801P_Seqlisting.txt; Size: 11,713 bytes; Created: Dec. 17, 2018.

### FIELD

The present disclosure relates generally to methods for inhibiting the growth of a tumor, treating cancer, and inducing or enhancing immune responses in a subject using at least two compositions. The present disclosure relates more specifically to compositions and methods comprising administering a first composition comprising a pathogen-associated molecular pattern molecule and a second composition comprising RNA (e.g., srRNA) encoding an immunomodulatory molecule such as IL-12.

### BACKGROUND

The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite.

Cancer includes a broad range of diseases and affects approximately one in four individuals worldwide. A CTL response is a key feature of effective cancer vaccines; effective CD4 T cell help is also likely to play a critical role in productive CD8 T cell activation and thus provide clinical benefit. The autologous dendritic cell (DC)-based vaccine Sipuleucel-T (PROVENGE®) was recently approved by the U.S. Food and Drug Administration (FDA) for the treatment of metastatic, castrate-resistant prostate cancer though the survival benefit associated with this treatment is a modest 4.1 months, leaving significant need for improvement (see, e.g., Kantoff, et al., New Engl. J. Med. 363(5):411 (2010)). The poxvirus-vector based vaccine ProstVac® VF also shows a significant survival benefit in Phase II (see, e.g., Kantoff, et al., J. Clin. Oncol. 28(7):1099 (2010)). Active immune therapies such as Sipuleucel-T and ProstVac® VF have generally been better tolerated than the chemotherapeutic regimens that comprise the current standard of care for castrate-resistant disease (see, e.g., Petrylak, et al., N. Engl. J. Med. 351(15):1513 (2004); Sylwester, et al., J. Exp. Med. 202(5):673 (2005)). These clinical successes demonstrate that the immune response can be harnessed in a cancer setting to provide improved patient outcomes and extended survival.

Cancer cells express antigens. Despite the presence of such antigens, tumors are generally not readily recognized and eliminated by the host, as evidenced by the development of disease. The inability of the immune system to protect against tumors may be due to mechanisms of evasion, active suppression, or sub-optimal activation of the response. Thus, there remains a need for improved agents and methods of activating the immune system to recognize cancers, including improved vaccines, for inducing a robust humoral and/or cell-mediated response for successful prevention and treatment of disease. In particular, considerable potential and need exists for improved cancer vaccine potency.

### SUMMARY OF THE INVENTION

One aspect of the present disclosure provides a method for inhibiting tumor growth in a subject, the method comprising (a) administering to the subject at least one dose of a first composition comprising a pathogen-associated molecular pattern (PAMP) molecule and (b) administering to the subject at least one dose of a second composition comprising a first RNA encoding at least one immunostimulatory molecule, thereby inducing an immune response.

Another aspect of the present disclosure provides a method for treating cancer in a subject, the method comprising (a) administering to the subject at least one dose of a first composition comprising a PAMP molecule; and (b) administering to the subject at least one dose of a second composition comprising a first RNA encoding at least one immunostimulatory molecule, thereby inducing an immune response.

In still another aspect, the present disclosure provides a method for inducing an immune response in a subject, the method comprising (a) administering to the subject at least one dose of a first composition comprising a PAMP molecule; and (b) administering to the subject at least one dose of a second composition comprising a first RNA encoding at least one immunostimulatory molecule, thereby inducing an immune response.

Various aspects of the aforementioned methods are contemplated wherein the PAMP, RNA, and other components and administration steps are provided. For example, in one aspect, a previous method is provided wherein the immunostimulatory molecule is IL-12. In another embodiment, the IL-12 is a single chain IL-12 (scIL-12). In one embodiment, the scIL-12 comprises p35-L-p40. In another embodiment, the scIL-12 comprises p40-L-p35.

In one aspect, an aforementioned method is provided wherein the PAMP molecule is an innate immune receptor agonist. In a related aspect, the innate immune receptor agonist is a glycan or a glycoconjugate. In various other aspects of the present disclosure, the PAMP molecule is a lipoprotein or lipopeptide, the PAMP molecule is a TLR agonist, the PAMP molecule is a TLR4 agonist, or the PAMP molecule is a TLR1, TLR2, TLR3, TLR4, TLR5, TLR6, TLR7, TLR8, TLR9, TLR10, TLR11, TLR12 or TLR13 agonist. In a related aspect, the TLR4 agonist is an aqueous or oil in water emulsion formulation of a monophosphoryl lipid A or the TLR4 agonist is an aqueous or oil in water emulsion formulation of glucopyranosyl lipid A (GLA).

The present disclosure contemplates numerous routes and timing of adminstrations of the compositions described herein. In one aspect, an aforementioned method is provided wherein the first composition and the second composition are administered by a route independently selected from the group consisting of intratumorally, intradermally, intravenously, subcutaneously, intranodally and intramuscularly. In a lreated aspect, the first and second compositions are administered intratumorally. In another aspect, an aforementioned method is provided wherein the first composition and the second composition are administered concurrently. In another aspect, an aforementioned method is provided wherein the first composition and the second composition are administered concurrently and at least two doses of each composition are administered. In another aspect, an aforementioned method is provided wherein the first composition and the second composition are administered sequentially. In still another aspect, an aforementioned method is provided wherein the first composition and the second composition are administered sequentially and at least two doses of each composition are administered. In yet another aspect, an aforementioned method is provided wherein the second composition is administered prior to administration of the first composition.

In another aspect, an aforementioned is method provided wherein the first composition and the second composition are administered by the same route. In yet another aspect, an aforementioned method is provided wherein the first composition and the second composition are administered by different routes. In still another aspect, an aforementioned method is provided wherein the first composition and the second composition are administered sequentially and by the same route. In another aspect, an aforementioned method is provided wherein the first composition and the second composition are administered sequentially and by different routes. In another aspect, an aforementioned method is provided wherein at least two doses of the first composition are administered or wherein at least two doses of the second composition are administered. In yet another aspect, an aforementioned method is provided wherein (a) two doses; (b) three doses; (c) four doses; or (d) five doses of the first composition are administered. In another aspect, an aforementioned method is provided wherein two doses of the first composition are administered prior to administration of the second composition.

As described herein, numerous RNAs are contemplated by the preent disclosure. In one aspect, an aforementioned method is provided wherein the RNA is a self-replicating RNA (srRNA). In a related aspect, the srRNA is derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

As also described herein numerous components may additionally comprise the compostion comprising the RNA. In one aspect, an aforementioned method is provided wherein the second composition comprises a cationic lipid, an ionizable lipid, a liposome, a nanoparticle, a lipid nanoparticle, a cochleate, a virosome, an immune-stimulating complex, a microparticle, a microsphere, a unilamellar vesicle, a multilamellar vesicle, an oil-in water emulsion, a water-in-oil emulsion, an emulsome, a polycationic peptide, or a cationic nanoemulsion. In a related aspect, the second composition comprises a lipid nanoparticle. In another aspect, an aforementioned method is provided wherein the second composition comprises a polyethylenimine derivative. In a related aspect, the polyethylenimine derivative comprises JETPEI. In still another aspect, an aforementioned method is provided wherein the second composition further comprises an adjuvant. In another aspect, an aforementioned method is provided wherein the second composition comprises a second, third or fourth RNA encoding an additional immunomodulatory molecule.

In yet another aspect of the present disclosure, an aforementioned method is provided further comprising administering one or more additional active agents or treatments. In a related aspect, the one or more additional active agents or treatments is selected from the group consisting of an immune checkpoint inhibitor, an antibody that activates a co-stimulatory pathway, a cancer chemotherapy, and radiation therapy.

Kits are prvided by the present disclosure. In one aspect, a kit is provided comprising (a) a first composition comprising PAMP molecule; and (b) a second composition comprising a first RNA encoding at least one immunostimulatory molecule.

Products comprising various compositions are also contemplated by the present disclosure. In aspect, a product comprising (a) a first composition comprising a PAMP molecule; and (b) a second composition comprising a first RNA encoding at least one immunostimulatory molecule, for use in a method selected from the group consisting of (i) inhibiting tumor growth in a subject, (ii) treating cancer in a subject, and (iii) inducing an immune response in a subject, is provided. In a related aspect, the immunostimulatory molecule is IL-12. In a related aspect, the IL-12 is a single chain IL-12 (scIL-12) or the scIL-12 comprises p35-L-p40 or. the scIL-12 comprises p40-L-p35.

In another aspect, an aforementioned product is provided wherein the PAMP molecule is an innate immune receptor agonist. In a related aspect, the innate immune receptor agonist is a glycan or a glycoconjugate. In a related aspect, the PAMP molecule is a lipoprotein or lipopeptide. In still another aspect, the PAMP molecule is a TLR agonist. In one aspect, the PAMP molecule is a TLR4 agonist. In still another aspect, the PAMP molecule is a TLR1, TLR2, TLR3, TLR4, TLR5, TLR6, TLR7, TLR8, TLR9, TLR10, TLR11, TLR12 or TLR13 agonist. In related aspects, the a PAMP molecule is an aqueous or oil in water emulsion formulation of glucopyranosyl lipid A (GLA).

In still another aspect, an aforementioned product is provided wherein the first composition and the second composition are administered by a route independently selected from the group consisting of intratumorally, intradermally, intravenously, subcutaneously, intranodally and intramuscularly. In one related aspect, the first and second compositions are administered intratumorally.

In various aspects, an aforementioned product is provided wherein the first composition and the second composition are administered concurrently; or wherein the first composition and the second composition are administered concurrently and at least two doses of each composition are administered; or wherein the first composition and the second composition are administered sequentially; or wherein wherein the first composition and the second composition are administered sequentially and at least two doses of each composition are administered; or wherein the second composition is administered prior to administration of the first composition. In still other aspects, an aforementioned product is provided wherein the first composition and the second composition are administered by the same route; or wherein the first composition and the second composition are administered by different routes; or wherein the first composition and the second composition are administered sequentially and by the same route; or wherein the first composition and the second composition are administered sequentially and by different routes.

In other aspects, an aforementioned product is provided wherein at least two doses of the first composition are administered or wherein at least two doses of the second composition are administered. In another aspect, an aforementioned product is provided wherein (a) two doses; (b) three doses; (c) four doses; or (d) five doses of the first composition are administered. In another aspect, an aforementioned product is provided wherein two doses of the first composition are administered prior to administration of the second composition.

Various RNAs may comprise the aforementioned products. In one aspect, an aforementioned product is provided wherein the RNA is a self-replicating RNA (srRNA). In a related aspect, the srRNA is derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

The products may also comprise one or more additional components as described herein. In one aspect, an aforementioned product is provided wherein the second composition comprises a cationic lipid, an ionizable lipid, a liposome, a nanoparticle, a lipid nanoparticle, a cochleate, a virosome, an immune-stimulating complex, a microparticle, a microsphere, a unilamellar vesicle, a multilamellar vesicle, an oil-in water emulsion, a water-in-oil emulsion, an emulsome, a polycationic peptide, or a cationic nanoemulsion. In a related aspect, the second composition comprises a lipid nanoparticle. In another aspect, an aforementioned product is provided wherein the second composition comprises a polyethylenimine derivative. In a related aspect, the polyethylenimine derivative comprises JETPEI.

In yet another aspect of the present disclosure, an aforementioned product is provided wherein the second composition further comprises an adjuvant. In another aspect, an aforementioned product is provided wherein the second composition comprises a second, third or fourth RNA encoding an additional immunomodulatory molecule.

In another aspect, an aforementioned product is provided further comprising one or more additional active agents or treatments. In a related aspect, the one or more additional active agents or treatments is selected from the group consisting of an immune checkpoint inhibitor, an antibody that activates a co-stimulatory pathway, a cancer chemotherapy, and radiation therapy.

In one aspect of the present disclosure, a method for inhibiting tumor growth in a subject is provided, the method comprising (a) administering intratumorally to the subject at least one dose of a first composition comprising GLA; and (b) administering intratumorally to the subject at least one dose of a second composition comprising a first RNA encoding IL-12, thereby inducing an immune response.

In one aspect of the present disclosure, a method for treating cancer in a subject is provided, the method comprising (a) administering intratumorally to the subject at least one dose of a first composition comprising GLA; and (b) administering intratumorally to the subject at least one dose of a second composition comprising a first RNA encoding IL-12, thereby inducing an immune response.

In one aspect of the present disclosure, a method for inducing an immune response in a subject is provided, the method comprising (a) administering intratumorally to the subject at least one dose of a first composition comprising GLA; and (b) administering intratumorally to the subject at least one dose of a second composition comprising a first RNA encoding IL-12, thereby inducing an immune response.

### BRIEF DESCRIPTION OF THE DRAWINGS

FIG. 1A is a diagram depicting alphavirus RNA replication cycle. Although significantly larger than and having numerous different properties from conventional cellular mRNA, the alphaviral genome functions as an mRNA, is 5′-capped and 3′ polyadenylated. Replication of the alphavirus self-replicating RNA yields high levels of a shorter, sub-genomic RNA species derived from the 3′ end of the RNA and driven from the 26S RNA dependent RNA polymerase promoter. FIG. 1B is a diagram depicting an illustrative RNA replicon derived from VEE. 5′ UTR, nsPs, 26S promoter, and 3′ UTR sequences taken from GenBank: L01443.1 (version 1, Gi 323714, updated Apr. 23, 2010), Venezuelan equine encephalitis virus strain TC-83 (attenuated). nsP—non-structural protein; sP—structural protein; SOI—sequence of interest.

FIG. 2 contains graphs illustrating the transfection efficiency (FIG. 2A) and GFP expression level (FIG. 2B) following transfection of BHK-21 cells with RNA using a commercially available transfection agent, such as Lipofectin.

FIG. 3 contains a graph demonstrating the use of the Quant-iT RiboGreen assay to quantify the lost fluorescence associated with complexation of RNA with a cationic polymer, such as jetPEI. Efficient complexation blocks binding of a fluorescent dye to RNA, indicated by a loss in mean fluorescence intensity.

FIG. 4 contains a graph comparing the expression of the sequence of interest (GFP) followed by transfection of BHK-21 with GFP-expressing RNA formulated with a cationic polymer (i.e. jetPEI) and a cationic lipid (Lipofectin) transfection reagent.

FIG. 5A and FIG. 5B are graphs demonstrating the stability of jetPEI-RNA complexes. BHK-21 cells were transfected with jetPEI-RNA complexes 4 hours after initial complexation, but no loss of transfection efficiency was observed.

FIG. 6A is a graph showing that IL12 can be detected at high levels in the serum of mice injected with 1 ug or 3 ug of IL12/srRNA formulated in jetPEI. FIG. 6B is a graph showing that the IL12 expressed by the jetPEI-srRNA in 6A is active and induces IFNγ production that can be detected in mouse serum.

FIG. 7A, 7B, 7C, 7D: Therapeutic Efficacy of G100±RNA/mIL12 in the B16 model. C57BL/6 mice (8-10 mice per group) were inoculated with 1.5×105 B16F10 cells, subcutaneously in the right flank, on Day 0. Seven days post-tumor challenge, tumors measured >4 mm2, and tumor-bearing mice were given intratumoral G100 (10 μg GLA in 2% SE), intratumoral naked RNA expressing mouse IL12 (nRNA/mIL12, 8 ug), intratumoral RNA/mIL12 formulated with jetPEI (fRNA/mIL12), G100+nRNA/mIL12, or G100+fRNA/mIL12. G100 administration was continued every 3-4 days thereafter, whereas RNA/mIL12 was administered once per week. Intratumoral treatments were stopped once tumors completely regressed. Tumor volume was measured 2-3 times a week and was calculated based on a modified ellipsoid formula: length×width2×π/6. A) Tumor volume of individual mice. B) Average tumor volume. C) Survival curve. D) Tumor volume of individual control and original surviving mice following tumor re-challenge. Error bars represent mean±SEM.

FIG. 8A, 8B, 8C: Therapeutic Efficacy of G100±RNA/mIL12 in the CT26 model. BALB/c (9-10 mice per group) were inoculated with 1.5×105 and 0.5×105 CT26 cells, subcutaneously in the right and left flank, respectively, on Day 0. Seven days post-tumor challenge, right flank tumors measured >4 mm2, and only right flank tumors were treated with intratumoral G100 (10 μg GLA in 2% SE), intratumoral naked RNA expressing mouse IL12 (nRNA/mIL12, 8 ug), intratumoral RNA/mIL12 formulated with jetPEI (fRNA/mIL12), G100+nRNA/mIL12, or G100+fRNA/mIL12. G100 administration was continued every 3-4 days thereafter, whereas RNA/mIL12 was administered once per week. Intratumoral treatments were stopped once tumors completely regressed. Tumor volume was measured 2-3 times a week and was calculated based on a modified ellipsoid formula: length×width2×π/6. A) Tumor volume of individual mice. B) Average tumor volume. C) Survival curve. Error bars represent mean±SEM.

FIG. 9A and FIG. 9B: Therapeutic Efficacy of G100±RNA/mIL12 in the 4T1 model. BALB/c (8 mice per group) were inoculated with 1.0×105 4T1 cells, subcutaneously in the right flank, on Day 0. Six days post-tumor challenge, tumors measured >4 mm2, and tumor-bearing mice were given intratumoral G100 (20 μg GLA in 2% SE), intratumoral ZVex expressing mIL12 (Z/mIL12), intramuscular RNA/mIL12 formulated with jetPEI (R/mIL12), G100+Z/mIL12, or G100+R/mIL12. G100 was administered again on Day 11, followed by 4T1 tumor resection on Day 12 (8A). G100 administration was continued thrice per week thereafter, intramuscularly, whereas RNA/mIL12 was administered once per week, until the end of the study. Tumor volume was measured 2-3 times a week and was calculated based on a modified ellipsoid formula: length×width2×π/6. A) Average tumor volume of mice, prior to tumor resection. B) Survival curve. Error bars represent mean±SEM.

### BRIEF DESCRIPTION OF THE SEQUENCE IDENTIFIERS

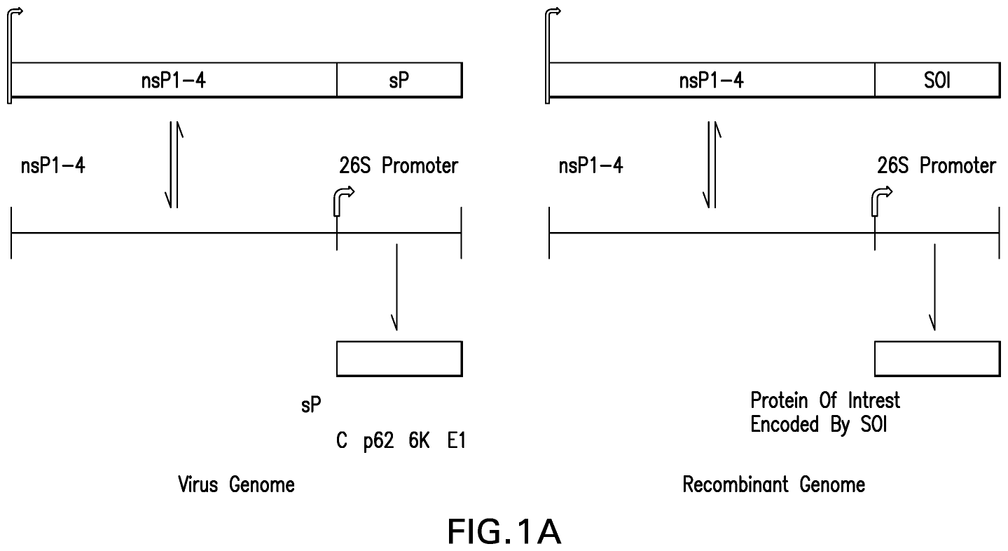

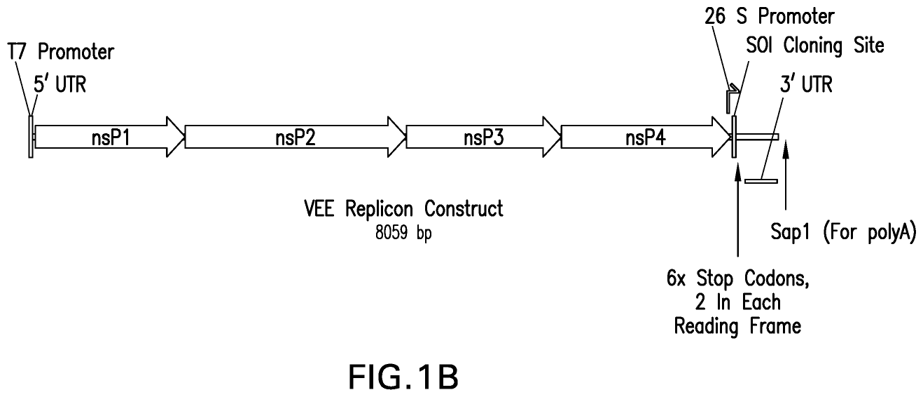

SEQ ID NO 1 is the polynucleotide sequence of an exemplary alphavirus replicon as depicted in FIG. 1B. 5′UTR: 1-44; REP (nsPl-4): 45-7523; Sub-genomic Promoter: 7501-7545; SOI (i.e. GFP): 7574-8293; 3′UTR: 8325-8441.

### DETAILED DESCRIPTION

The present disclosure is based in part on the discovery that administering a PAMP such as a TLR4 agonist (e.g., glucopyranosyl lipid A (GLA)) in combination with an RNA encoding an immunomodulatory agent such as IL-12 surprisingly results in complete regression of tumors and significant improvement in survival. As such, the present disclosure provides a highly effective and potentially long-lasting cancer treatment.

### Pathogen-Associated Molecular Pattern (PAMP) Molecules

The discovery of pattern recognition receptors (PRRs) revolutionized the prevailing view of innate immunity, revealing its intimate connection with adaptive immunity and generation of effector and memory T- and B-cell responses (Reviewed in Munoz-Wolf, N. and Lavelle EC., Methods Mol Biol. 2016; 1417:1-43). Among the PRRs, families of Toll-like receptors (TLRs), C-type lectin receptors (CLR), retinoic acid-inducible gene-I (RIG-I)-like receptors (RLRs) and nucleotide-binding domain, leucine-rich repeat-containing protein receptors (NLRs), along with a number of cytosolic DNA sensors and the family of absent in melanoma (AIM)-like receptors (ALRs), have been characterized.

Pathogen-associated molecular pattern molecules (PAMP, PAMPs or PAMP molecule(s)) activate innate immune responses, protecting the host from infection, by identifying some conserved non-self molecules. Bacterial lipopolysaccharides (LPSs) are considered to be the prototypical class of PAMPs. LPSs are specifically recognized by TLR4, a recognition receptor of the innate immune system. Other PAMPs include bacterial flagellin (recognized by TLR5), lipoteichoic acid from gram-positive bacteria (recognized by TLR2), peptidoglycan (recognized by TLR2), and nucleic acid variants normally associated with viruses, such as double-stranded RNA (dsRNA), recognized by TLR3 or unmethylated CpG motifs, recognized by TLR9.

As described herein, PAMPs activate innate immune receptors and therefore any compositions or methods comprising one or more innate immune receptor agonist are contemplated. Many PAMPs are glycoconjugates (e.g., bacterial lipo-oligosaccharides) or glycan-based polymers (e.g., bacterial peptidoglycans), including bacterial DNA or viral RNA (which are (deoxy)ribose-based polymers). The innate immune system also recognizes “danger-associated molecular patterns” (DAMPs; Matzinger P Science. 2002 Apr. 12; 296(5566):301-5; Chen and Nunez, Nat Rev Immunol. 2010 December; 10(12):826-37), molecules released during tissue damage, such as heat-shock proteins, high mobility group box 1 (Lotze and Tracey, Nat Rev Immunol. 2005 April; 5(4):331-42), hyaluronan (HA) fragments (Taylor and Gallo, FASEB J. 2006 January; 20(1):9-22), glycosaminoglycan (GAG)-bearing matrix proteoglycans (Moreth et al., J Clin Invest. 2010 December; 120(12):4251-72) and certain crystals (Martinon et al., Annu Rev Immunol. 2009; 270:229-65.), all of which originate from damaged host cells or matrices. Signals initiated by DAMPs and PAMPs are transduced via similar pathways, activating innate immune inflammatory responses.

The terms glycan and polysaccharide are defined by IUPAC as synonyms meaning “compounds consisting of a large number of monosaccharides linked glycosidically”. However, in practice the term glycan may also be used to refer to the carbohydrate portion of a glycoconjugate, such as a glycoprotein, glycolipid, or a proteoglycan, even if the carbohydrate is only an oligosaccharide. Glycans usually consist solely of O-glycosidic linkages of monosaccharides. For example, cellulose is a glycan (or, to be more specific, a glucan) composed of β-1,4-linked D-glucose, and chitin is a glycan composed of β-1,4-linked N-acetyl-D-glucosamine. Glycans can be homo- or heteropolymers of monosaccharide residues, and can be linear or branched. Glycans can be found attached to proteins as in glycoproteins and proteoglycans. In general, they are found on the exterior surface of cells. O- and N-linked glycans are very common in eukaryotes but may also be found, although less commonly, in prokaryotes.

Lipoproteins, lipopeptides and lipopolysaccharides that activate an innate immune receptor are also contemplated for use in the disclosed compositions and methods.

Toll-like receptors are so named because of their homology to a gene named Toll, first identified in Drosophila. TLRs were the first PRRs to be discovered and have come to represent the archetype of innate immune recognition receptors. Humans have 10 TLRs, each with a leucine-rich repeat (LRR) domain involved in recognition of microbial components, and an intracytoplasmic TIR domain involved in signaling into the cell. TLRs associate with a variety of adaptor molecules that help to convert recognition of microbes into a signal, which activates specific gene transcription within the cell. TLRs play a vital role in activating immune responses. TLRs recognize conserved pathogen-associated molecular patterns (PAMPs) expressed on a wide array of microbes, as well as endogenous DAMPs released from stressed or dying cells. TLR1, -2, -4, -5, -6, and -10 are expressed on the cell surface, whereas TLR3, -7, -8, and -9 are situated on endosomal membranes within the cell. An overview of TLRs, agonists, and use in therapy is provided in Kaczanowska S. et al., J Leukoc Biol. 2013 June; 93(6): 847-863.

Exemplary TLR agonists for use in the compositions and methods described herein include, but are not limited to, triacylated lipoproteins, lipoteichoic acid, peptidoglycans (TLR1-TLR2); diacylated lipopeptides, HSPs, HMGB1, uric acid, fibronectin, ECM proteins, and Pam3CSK4 (TLR2-TLR6); dsRNA and Poly I:C (TLR3); LPS, lipoteichoic acid, β-defensin 2, fibronectin EDA, HMGB1, snapin, tenascin C (TLR4); flagellin (TLR5); ssRNA, CpG, Poly G10, Poly G3, resiquimod, imiquimod, 3M052 (TLR7-TLR8); unmethylated CpG DNA (TLR9); and Pam3CSK4, PamCysPamSK4 (TLR10). In certain embodiments, a TLR agonist is an antibody or antigen-binding fragement thereof that binds to and is an agonist for a particular TLR receptor.

In exemplary embodiments, agonists of TLR4 are contemplated. By way of example, the TLR4 agonist is a monophosphoryl lipid A or glucopyranosyl lipid A (GLA) (e.g., as described in U.S. Pat. No. 8,273,361). In certain embodiments, the TLR4 agonist is an aqueous or oil in water emulsion formulation of a monophosphoryl lipid A or an aqueous or oil in water emulsion formulation of glucopyranosyl lipid A (GLA).

In one embodiment, the PAMP that may be used in the compositions described herein is identified by chemical formula (I) and referred to as glucopyranosyl lipid A (GLA):

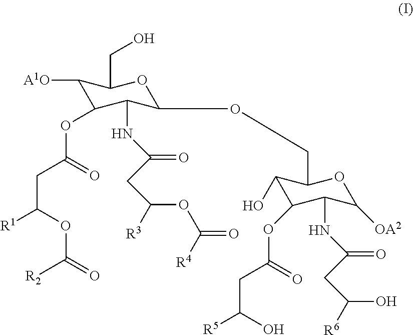

wherein the moieties A1 and A2 are independently selected from the group of hydrogen, phosphate, and phosphate salts. Sodium and potassium are exemplary counterions for the phosphate salts. The moieties R1, R2, R3, R4, R5, and R6 are independently selected from the group of hydrocarbyl having 3 to 23 carbons, represented by C3-C23. For added clarity it will be explained that when a moiety is “independently selected from” a specified group having multiple members, it should be understood that the member chosen for the first moiety does not in any way impact or limit the choice of the member selected for the second moiety. The carbon atoms to which R1, R3, R5 and R6 are joined are asymmetric, and thus may exist in either the R or S stereochemistry. In one embodiment all of those carbon atoms are in the R stereochemistry, while in another embodiment all of those carbon atoms are in the S stereochemistry.

“Hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, i.e., to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl”, respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The PAMP of formula (I) may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. Certain of the TLR4 agonists of formulat (I) may also be obtained commercially. A preferred PAMP E1 in combination with E10, below.

In various embodiments, the PAMP has the chemical structure of formula (I) but the moieties A1, A2, R1, R2, R3, R4, R5, and R6 are selected from subsets of the options previously provided for these moieties, wherein these subsets are identified below by E1, E2, etc.

E1: A1 is phosphate or phosphate salt and A2 is hydrogen.

E2: R1, R3, R5 and R6 are C3-C21 alkyl; and R2 and R4 are C5-C23 hydrocarbyl.

E3: R1, R3, R5 and R6 are C5-C17 alkyl; and R2 and R4 are C7-C19 hydrocarbyl.

E4: R1, R3, R5 and R6 are C7-C15 alkyl; and R2 and R4 are C9-C17 hydrocarbyl.

E5: R1, R3, R5 and R6 are C9-C13 alkyl; and R2 and R4 are C11-C15 hydrocarbyl.

E6: R1, R3, R5 and R6 are C9-C15 alkyl; and R2 and R4 are C11-C17 hydrocarbyl.

E7: R1, R3, R5 and R6 are C7-C13 alkyl; and R2 and R4 are C9-C15 hydrocarbyl.

E8: R1, R3, R5 and R6 are C11-C20 alkyl; and R2 and R4 are C12-C20 hydrocarbyl.

E9: R1, R3, R5 and R6 are C11 alkyl; and R2 and R4 are C13 hydrocarbyl.

E10: R1, R3, R5 and R6 are undecyl and R2 and R4 are tridecyl.

In certain embodiments, E1 may be combined with each of E2 through E10. The hydrocarbyl groups of E2 through E9 may be alkyl groups, preferably straight chain alkyl groups. In certain embodiments, E1 is combined with each of E2 through E10 and each of E2 through E9 are alkyl groups. In certain embodiments, E1 is combined with each of E2 through E10 and each of E2 through E9 are straight chain alkyl groups. The PAMP of formula (I) may be formulated into a pharmaceutical composition, optionally with a co-adjuvant, each as discussed below. In this regard reference is made to U.S. Patent Publication No. 2008/0131466 that provides formulations, such as aqueous formulation (AF) and stable emulsion formulations (SE) for GLA, wherein these formulations may be used for any of the PAMPs of formula (I).

In another embodiment, the PAMP is a synthetic lipid A type molecule as described in PCT International publication No. WO 2010/141861, and has a structure selected from the following chemical formula (II):

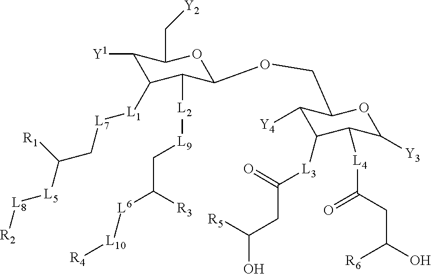

or a pharmaceutically acceptable salt thereof, wherein: L1, L2, L3, L4, L5 and L6 are the same or different and are independently selected from —O—, —NH—, and —(CH2)-; L7, L8, L9 and L10 are the same or different, and at any occurrence may be either absent or —C(═O)—; Y1 is an acid functional group; Y2 and Y3 are the same or different and are each independently selected from —OH, —SH, and an acid functional group; Y4 is —OH or —SH; R1, R3, R5 and R6 are the same or different and are each independently selected from the group of C8-C13 alkyl; and R2 and R4 are the same or different and are each independently selected from the group of C6-C11 alkyl.

In certain embodiments, the PAMP of formula (I) may be formulated into a pharmaceutical (or adjuvant composition), optionally with a co-adjuvant as described above, each as discussed below or any other adjuvant described herein or available in the art. In this regard reference is made to U.S. Patent Publication No. 2008/0131466 that provides formulations, such as aqueous formulation (AF) and stable emulsion formulations (SE) for GLA, which formulations may be used with respect to any of the PAMPs of formula (I).

In still another embodiment, the compounds described in WO 2010/141861 are contemplated. In one embodiment, Compound IX of WO 2010/141861 is contemplated. Compound IX has the structure shown in the following chemical formula:

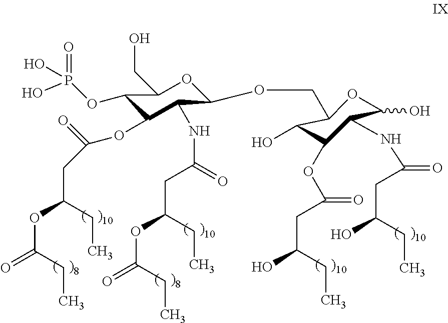

### RNA

The compositions containing RNA for use in the methods according to the disclosure comprises at least one RNA. The at least one RNA of the RNA containing composition may be selected from the group consisting of chemically modified or unmodified RNA, single-stranded or double-stranded RNA, coding or non-coding RNA, mRNA, oligoribonucleotide, viral RNA, retroviral RNA, self-replicating (replicon) RNA (srRNA), tRNA, rRNA, immunostimulatory RNA, microRNA, siRNA, small nuclear RNA (snRNA), small-hairpin (sh) RNA riboswitch, RNA aptamer, RNA decoy, antisense RNA, a ribozyme, or any combination thereof. In specific embodiments, the at least one RNA of the compositions comprising RNA is a coding RNA.

RNA is the usual abbreviation for ribonucleic acid. It is a nucleic acid molecule, i.e. a polymer consisting of nucleotide monomers. These nucleotides are usually adenosine monophosphate (AMP), uridine monophosphate (UMP), guanosine monophosphate (GMP) and cytidine monophosphate (CMP) monomers or analogues thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA (also called pre-mRNA, precursor mRNA or heterogeneous nuclear RNA) which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) tail.

In addition to messenger RNA, several non-coding types of RNA exist which may be involved in regulation of transcription and/or translation, and immunostimulation. Within the present invention the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

5′-CAP-Structure: A 5′-CAP is typically a modified nucleotide (CAP analogue), particularly a guanine nucleotide, added to the 5′ end of an mRNA molecule. In certain embodiments, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present invention to modify the RNA sequence of the present disclosure. Further modified 5′-CAP structures which may be used in the context of the present invention are CAP1 (additional methylation of the ribose of the adjacent nucleotide of m7GpppN), CAP2 (additional methylation of the ribose of the 2^{nd} nucleotide downstream of the m7GpppN), CAP3 (additional methylation of the ribose of the 3^{rd} nucleotide downstream of the m7GpppN), CAP4 (additional methylation of the ribose of the enucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In the context of the present disclosure, a 5′ cap structure may also be formed in chemical RNA synthesis or RNA in vitro transcription (co-transcriptional capping) using cap analogues, or a cap structure may be formed in vitro using capping enzymes (e.g., commercially available capping kits)

A cap analogue refers to a non-polymerizable di-nucleotide that has cap functionality in that it facilitates translation or localization, and/or prevents degradation of the RNA molecule when incorporated at the 5′ end of the RNA molecule. Non-polymerizable means that the cap analogue will be incorporated only at the 5′terminus because it does not have a 5′ triphosphate and therefore cannot be extended in the 3′ direction by a template-dependent RNA polymerase.

Cap analogues include, but are not limited to, a chemical structure selected from the group consisting of m7GpppG, m7GpppA, m7GpppC; unmethylated cap analogues (e.g., GpppG); dimethylated cap analogue (e.g., m2,7GpppG), trimethylated cap analogue (e.g., m2,2,7GpppG), dimethylated symmetrical cap analogues (e.g., m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7(10):1486-95).

Further cap analogues have been described previously (U.S. Pat. No. 7,074,596, WO 2008/016473, WO 2008/157688, WO 2009/149253, WO 2011/015347, and WO 2013/059475). The synthesis of N⁷-(4-chlorophenoxyethyl) substituted dinucleotide cap analogues has been described recently (Kore et al. (2013) Bioorg. Med. Chem. 21(15): 4570-4).

A poly(A) tail also called “3′-poly(A) tail” or “Poly(A) sequence” is typically a long homopolymeric sequence of adenosine nucleotides of up to about 400 adenosine nucleotides, e.g. from about 25 to about 400, from about 50 to about 400, from about 50 to about 300, from about 50 to about 250, or from about 60 to about 250 adenosine nucleotides, added to the 3′ end of an mRNA. In certain embodiments of the present disclosure, the poly(A) tail of an mRNA or srRNA is derived from a DNA template by RNA in vitro transcription. Alternatively, the poly(A) sequence may also be obtained in vitro by common methods of chemical synthesis without being necessarily transcribed from a DNA-progenitor. Moreover, poly(A) sequences, or poly(A) tails may be generated by enzymatic polyadenylation of the RNA.

A stabilized nucleic acid, typically, exhibits a modification increasing resistance to in vivo degradation (e.g. degradation by an exo- or endo-nuclease) and/or ex vivo degradation (e.g. by the manufacturing process prior to composition administration, e.g. in the course of the preparation of the composition to be administered). Stabilization of RNA can, e.g., be achieved by providing a 5′-CAP-Structure, a poly(A) tail, or any other UTR-modification. Stabilization can also be achieved by backbone-modification (e.g., use of synthetic backbones such as phosphorothioate) or modification of the G/C-content or the C-content of the nucleic acid. Various other methods are known in the art and conceivable in the context of the invention, to stabilize or otherwise improve the function of the nucleic acid. Provided herein, therefore, are polynucleotides which have been designed to improve one or more of the stability and/or clearance in tissues, receptor uptake and/or kinetics, cellular access, engagement with translational machinery, RNA half-life, translation efficiency, immune evasion, immune induction (for vaccines), protein production capacity, secretion efficiency (when applicable), accessibility to circulation, protein half-life and/or modulation of a cell's status, function and/or activity. See for example WO2017049275, WO2016011306, WO2016100812.

A 5′-UTR is typically understood to be a particular section of RNA. It is located 5′ of the open reading frame of the mRNA. In the case of srRNA, the open reading frame encodes the viral non-structural proteins while the sequence of interest is encoded in the subgenomic fragment of the viral RNA. Thus, the 5′UTR is upstream of nsP1 open reading frame (see e.g., FIG. 1B). In addition, the subgenomic RNA of the srRNA has a 5′UTR. Thus the subgenomic RNA containing a sequence of interest encoding a protein of interest contains a 5′UTR. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present disclosure, a 5′UTR corresponds to the sequence of a mature mRNA or srRNA which is located between the 5′-CAP and the start codon. In one embodiment, the 5′-UTR corresponds to the sequence which extends from a nucleotide located 3′ to the 5′-CAP, and in certain embodiments from the nucleotide located immediately 3′ to the 5′-CAP, to a nucleotide located 5′ to the start codon of the protein coding region and in some cases to the nucleotide located immediately 5′ to the start codon of the protein coding region. The nucleotide located immediately 3′ to the 5′-CAP of a mature mRNA or srRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a NYESO1 gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

Generally, the term “3′-UTR” refers to a part of the nucleic acid molecule which is located 3′ (i.e. “downstream”) of an open reading frame and which is not translated into protein. Typically, a 3′-UTR is the part of an RNA which is located between the protein coding region (open reading frame (ORF) or coding sequence (CDS)) and the poly(A) sequence of the mRNA. In the context of the present disclosure, the term 3′-UTR may also comprise elements, which are not encoded in the template, from which an RNA is transcribed, but which are added after transcription during maturation, e.g. a poly(A) sequence. A 3′-UTR of the RNA is not translated into an amino acid sequence.

With respect to srRNA, the 3′-UTR sequence is generally encoded by the viral genomic RNA, which is transcribed into the respective mRNA during the gene expression process. The genomic sequence is first transcribed into pre-mature mRNA. The pre-mature mRNA is then further processed into mature mRNA in a maturation process. This maturation process comprises 5′capping. In the context of the present invention, a 3′-UTR corresponds to the sequence of a mature mRNA or srRNA (and the srRNA subgenomic RNA), which is located between the stop codon of the protein coding region, preferably immediately 3′ to the stop codon of the protein coding region for the sequence of interest, and the poly(A) sequence of the mRNA. The term “corresponds to” means that the 3′-UTR sequence may be an RNA sequence, such as in the mRNA sequence used for defining the 3′-UTR sequence, or a DNA sequence, which corresponds to such RNA sequence. In the context of the present invention, the term “a 3′-UTR of a gene”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence (both sense and antisense strand and both mature and immature) of the 3′-UTR.

According to certain embodiments of the disclosure, the RNAs for use in the compositions and methods herein comprise an RNA comprising at least one coding region encoding at least one peptide or protein. In one embodiment, the coding RNA is selected from the group consisting of mRNA, viral RNA, retroviral RNA, and self-replicating RNA.

In certain embodiments, the RNA contemplated for use in the regimens described here is a self-replicating RNA (srRNA). Self-replicating RNA molecules replicate in host cells leading to an amplification of the amount of RNA encoding the desired gene product. This can enhance efficiency of RNA delivery and expression of the encoded gene products. See, e.g., Johanning, F. W., et al., Nucleic Acids Res., 23(9):1495-1501 (1995); Khromykh, A. A., Current Opinion in Molecular Therapeutics, 2(5):555-569 (2000); Smerdou et al., Current Opinion in Molecular Therapeutics, 1(2):244-251 (1999). Self-replicating RNAs have been produced as virus particles and as free RNA molecules (Liljestrom P. and Garoff H. 1991 Biotechnology. December; 9(12):1356-61). However, free RNA molecules are rapidly degraded in vivo, and most RNA-based vaccines that have been tested have had limited ability to provide antigen at a dose and duration required to produce a strong, durable immune response. See, e.g., Probst et al., Genetic Vaccines and Therapy, 4:4; doi:10.1186/1479-0556-4-4 (2006).

While the genomic alphavirus RNA functions as an mRNA, srRNA replicons as used herein are very different from typical mRNA for at least the following reasons: 1) mRNA encodes a single protein—srRNA replicons herein encode multiple viral proteins and protein of interest; 2) srRNA is usually very large ˜9-11 kb while mRNAs are typically ˜2 kb; 3) srRNA has different requirements for production and delivery.

In certain embodiments, one illustrative srRNA is a viral replicon. srRNA for use in the methods described herein can be derived from either positive- or negative-strand RNA viruses. RNA replicons are self-replicating RNAs that are able to drive high level, cytosolic expression of recombinant sequences of interest. In certain embodiments, the viral replicon sequence is derived from a virus in the Togaviridae family. The Togaviridae family includes the alphaviruses and rubivirus (rubella). (See e.g., McCullough at al., Molecular Therapy—Nucleic Acids 2014 volume 3 page e173). An illustrative RNA replicon derived from Venezuelan equine encephalitis virus (VEE) is shown in FIG. 1. Other Alphavirus replicons are also contemplated herein, including, but not limited to, RNA replicons from Eastern Equine Encephalitis Virus (EEE), Venezuelan Equine Encephalitis Virus (VEE), Everglades Virus, Mucambo Virus, Ockelbo virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Aura virus, Babanki Virus, Kyzylagach Virus, Highlands J virus, Fort Morgan Virus, Ndumu Virus, Buggy Creek Virus and any other virus classified by the International Committee on Taxonomy of Viruses (ICTV) as an alphavirus, as well as subgroups thereof as are known in the art. Additional Alphavirus species from which RNA replicons can be used include Salmon pancreatic disease virus, Sleeping Disease virus, Southern elephant seal virus, Tonate virus. In certain embodiments, RNA replicons from an alphavirus species with attractive properties (e.g. SPDV which is adapted to very low temperatures, or others alphavirus species with high activity in insect cells and thus adapted to modest temperatures) are selected for use herein. Other RNA virus replicons are also contemplated for use herein.

An alphaviral RNA replicon generally comprises the 4 genes encoding a full-length replicase polyprotein (REP) (which is comprised of nsP1, nsP2, nsP3 and nsP4) and optionally, the alphavirus 5′ and or 3′ non-coding sequences (5′UTR, 3′UTR) which contain cis-acting elements including Conserved Sequence Elements (see for example, Journal of Virology, 1990, p. 1639-1647). The genes encoding the nonstructural proteins comprise sequences necessary for replication and the replicase (Rep) gene.

In certain embodiments, an alphavirus packaging signal can be included.

In certain embodiments, a hepatitis delta virus ribozyme is included at the 3′ end following the poly-A stretch. This acts to process the RNA transcripts to unit lengths in a self-cleavage reaction, the end result being an RNA with a clean poly-A stretch end.

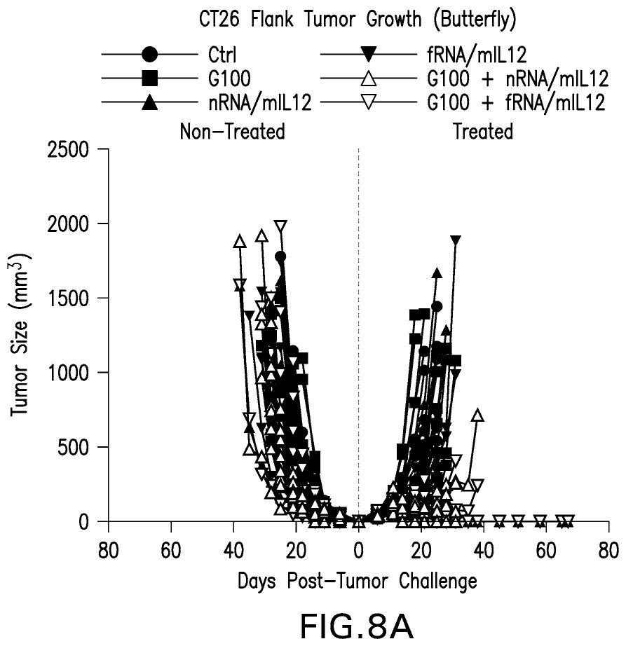

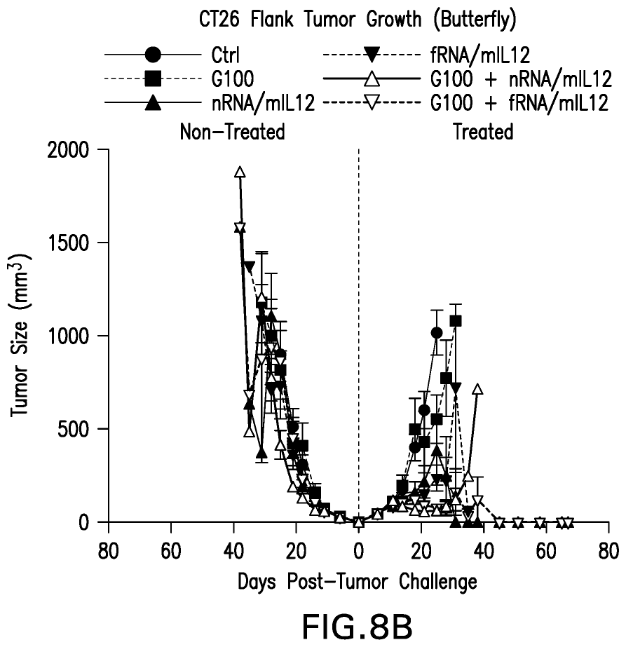

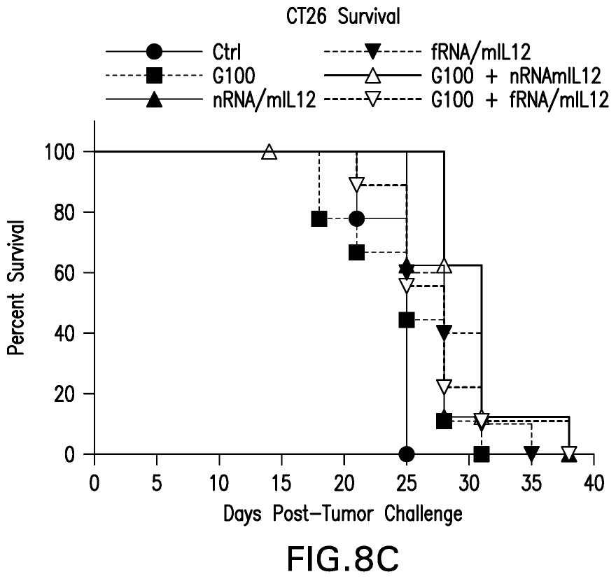

In another embodiment, translation of the replicase is achieved through a 2A endoprotease cleavage immediately at the beginning of the replicase (see e.g., FIG. 8D of WO2017/083356). This requires translational read-through from the 5′ end of the transcript, which is achieved by site-directed mutations that remove (mutate) stop codons in the sequence upstream of the replicase gene. As would be understood by the skilled person, the codons are not generally “removed” but are mutated to code for something other than a stop codon.

The RNA constructs of the present disclosure comprise a sequence of interest (SOI). The SOI useful in the RNAs here are the same as those described elsewhere herein in further detail. SOIs are positioned in such a manner within the RNA construct such that it is expressed in a target cell. In an srRNA construct, the sequence of interest (SOI) is typically inserted in place of Alphavirus structural protein genes (see e.g., FIG. 1A and FIG. 1B SOI downstream of either NSP4 or REP). In certain embodiments of the methods herein, the SOI in the composition comprising RNA encode the same protein of interest as expressed in the retroviral vector preparations, or are a variant thereof having activity that is not significantly reduced as compared to a reference SOI. However, in certain embodiments, the composition comprising RNA may comprise one or more different SOI from the retroviral vector preparations. For example, in certain embodiments, the composition comprising RNA comprises a SOI encoding one or more immunomodulatory agents.

In one embodiment, the compositions comprising RNA for use in the methods herein comprise a first RNA comprising a sequence of interest. In one embodiment, the RNA sequence of interest encodes an antigen. In another embodiment, the RNA sequence of interest encodes an immunomodulatory molecule. In certain embodiments, the compositions comprising RNA for use in the methods herein comprise a first and a second RNA, each comprising a unique sequence of interest. In another embodiment, the first RNA comprises a sequence of interest encoding an antigen and the second RNA comprises a sequence of interest encoding an immunomodulatory molecule. In another embodiment, the compositions comprising RNA for use in the methods herein comprise a first RNA, a second RNA, and a third RNA, each comprising a unique sequence of interest. In another embodiment, the compositions comprising RNA for use in the methods herein comprise a first RNA, a second RNA, a third RNA, and a fourth RNA, each comprising a unique sequence of interest. In one embodiment, the first, second, third and fourth RNAs each comprise a unique sequence of interest, three of which encode antigens, one of which encodes an immunomodulatory molecule.

In another embodiment, the compositions comprising RNA for use in the methods herein comprise a first RNA, a second RNA, a third RNA, a fourth RNA, and a fifth RNA, each comprising a unique sequence of interest (and thus each encoding a unique protein of interest). In another embodiment, the compositions comprising RNA for use in the methods herein comprise a first RNA, a second RNA, a third RNA, a fourth RNA, a fifth RNA and a sixth RNA, each comprising a unique sequence of interest. In other embodiments, the compositions comprising RNA for use in the methods herein comprise a first, second, third, fourth, fifth, sixth, seventh, eighth, ninth, tenth, eleventh, twelfth, or more different RNAs, each comprising a unique sequence of interest. In this regard, the sequence of interest of each of the RNAs in a given composition to be used in the methods herein may encode a unique protein of interest. In certain embodiments, the different RNAs in a given composition may encode variants of the same protein. In certain embodiments, one or more of the RNAs in the composition encodes one or more neoantigens.

In certain embodiments of the invention the at least one RNA of the RNA containing composition codes for at least one cytokine and/or for at least one chemokine and/or for at least one suicide gene product, and/or at least one immunogenic protein or peptide (antigen) and/or for at least one cell death/apoptosis inducer and/or for at least one angiogenesis inhibitor and/or for at least one heat shock protein and/or for at least one tumor antigen and/or for at least one beta-catenin inhibitor and/or for at least one activator of the STING (stimulator of interferon genes) pathway and/or at least one immune checkpoint modulator and/or at least one antibody, and/or at least one dominant negative receptor, and/or at least one decoy receptor, and/or at least one inhibitor of myeloid derived suppressor cells (MDSCs), and/or at least one IDO pathway inhibitor, and/or at least one protein or peptide that bind inhibitors of apoptosis, or fragments or variants thereof as described in more detail elsewhere herein.

Any method known in the art for making RNA is contemplated herein for making the RNAs. Illustrative methods for making RNA include but are not limited to, chemical synthesis and in vitro transcription.

In certain embodiments, the RNA for use in the methods herein is chemically synthesized. Chemical synthesis of relatively short fragments of oligonucleotides with defined chemical structure provides a rapid and inexpensive access to custom-made oligonucleotides of any desired sequence. Whereas enzymes synthesize DNA and RNA only in the 5′ to 3′ direction, chemical oligonucleotide synthesis does not have this limitation, although it is most often carried out in the opposite, i.e. the 3′ to 5′ direction. In certain embodiments, the process is implemented as solid-phase synthesis using the phosphoramidite method and phosphoramidite building blocks derived from protected nucleosides (A, C, G, and U), or chemically modified nucleosides.

In some embodiments, modifications are included in the modified nucleic acid or in one or more individual nucleoside or nucleotide. For example, modifications to a nucleoside may include one or more modifications to the nucleobase, the sugar, and/or the internucleoside linkage. In some embodiments having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 1-(2,2,2-trifluoroethyl)-pseudouridine-MP, 2-amino-adenine-MP, xanthosine-MP, 5-bromo-cytidine-MP, 5-aminoallyl-cytidine-MP, or 2-aminopurine-riboside-MP.

In certain embodiments having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 5-bromo-cytidine-MP, 5-aminoallyl-cytidine-MP, or 2-aminopurine-riboside-MP.

In other embodiments having at least one modification, the polynucleotide includes a backbone moiety containing the nucleobase, sugar, and internucleoside linkage of: pseudouridine-alpha-thio-MP, 1-methyl-pseudouridine-alpha-thio-MP, or 5-bromo-cytidine-MP. Nucleoside and nucleotide modifications contemplated for use in the present disclosure are known in the art. Exemplary nucleoside/nucleotide modifications contemplated for use in the present disclosure include those as described for example in published international applications WO2018005974; WO2015089511; and U.S. Pat. No. 9,657,295.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

In certain embodiments, RNA is made using in vitro transcription. The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which in certain embodiments is a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In one embodiment of the present disclosure, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art (see, e.g., Current Protocols in Molecular Biology, (2017) John Wiley & Sons, Inc.; RNA Methods and Protocols, Henrik Nielsen (Ed.), ISBN 978-1-59745-248-9; Geall et al. (2013) Semin. Immunol. 25(2): 152-159; Brunelle et al. (2013) Methods Enzymol. 530:101-14). Reagents used in said method typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) optionally a cap analogue as defined above (e.g. m7G(5′)ppp(5′)G (m7G)); 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl₂, which supplies Mg²⁺ ions as a co-factor for the polymerase; 8) a buffer to maintain a suitable pH value, which can also contain antioxidants (e.g. DTT), and/or polyamines such as spermidine at optimal concentrations.

### Delivery of RNA/RNA Formulations

RNA may be formulated for delivery to subjects in need thereof according to various embodiments of the invention. In some embodiments, the RNA molecule is part of a delivery system, e.g., a non-viral delivery system. Delivery systems suitable for use in the methods of the present disclosures and provided herein include any of those known in the art. See, e.g., Zhang et al., 2013, supra. In exemplary embodiments, the delivery system is a liposome, an aptamer complex, a nanoparticle, or a dendrimer. Accordingly, the disclosure provides such delivery systems. The invention provides a liposome, an aptamer complex, a nanoparticle, a dendrimer, or an extracellular vesicle comprising an RNA molecule or a recombinant expression vector, among other possibilities, described herein. Other delivery systems or formulations for delivery of RNA as described herein include compositions comprising a cationic lipid, an ionizable lipid, a liposome, a nanoparticle, a lipid nanoparticle, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, or a cationic nanoemulsion. In certain embodiments, the formulations for delivery of RNA as described herein include compositions comprising multilamellar lipid vesicles. In one embodiment, the multilamellar lipid vesicles have covalent crosslinks between lipid bilayers, where at least two lipid bilayers in the multilamellar lipid vesicle are covalently crosslinked to each other through headgroups that react with covalent crosslinkers to form the covalent crosslinks between lipid bilayers. See for example, WO2011115684.

In exemplary embodiments, the RNA molecule is part of an aptamer-oligonucleotide conjugate. Such conjugates are described in the art. See, e.g., Liu et al., Cancer investigation 30:577-582 (2012). In exemplary embodiments, the RNA molecule is part of a lipid-based delivery system, such as siPORT, MaxSuppressor, LipoTrust, TransMessenger, and Lipofectamine, each of which are commercially-available and described in the art. See, e.g., Wu et al., Molecular Pharmaceutics 8:1381-1389 (2011); Craig et al., Leukemia 26:2421-2424 (2012); Trang et al., Mol Ther 19:1116-1122 (2011); Akao et al., Cancer Gene Ther 17:398-408 (2010). In exemplary embodiments, the RNA molecule is part of a lipid-based delivery system that is not commercially available, e.g., a 98N₁₂-5 delivery system (Akinc et al., Nature Biotechnology 26:561-569 (2008); a 1,2-Di-O-octadecenyl-3-trimethylammonium propane (DOTMA):cholesterol:D-alpha-tocopheryl polyethylene glycol 1000 succinate (TPGS) lipoplex (Wu et al., Molecular Pharmaceutics 8:1381-1389 (2011); a dimethyldioctadecylammonium bromide (DDAB):cholesterol:TPGS lipoplex (Piao et al., Molec Therapy 20:1261-1269 (2012); a targeted liposome-hyaluronic acid (LPH) nanoparticle (Chen et al., Mole Ther 18:1650-1656 (2010); and Liu et al., Molec Pharmaceutics 8:250-259 (2011)); an nanotransporter interfering nanoparticle-7 (iNOP-7) (Su et al., Nucleic Acids Res 39:e38 (2011)); a DC-6-140-DOPE-cholesterol liposome (Rai et al., Molec Cancer Ther 10:1720-1727 (2011)); or a solid lipid nanoparticle (Shi et al., Systemic Delivery of microRNA-34A for Cancer Stem Cell Therapy, Angewandte Chemie (2013)).

In exemplary embodiments, the RNA molecule is part of a polyethyleneimine (PEI) conjugate (Ibrahim et al., Cancer Research 71:5214-5224 (2011)); a polyurethane-short branch PEI conjugate (Chiou et al., J Control Release 159:240-250 (2012); a dendrimer (e.g., a poly(amidoamine) dendrimer (Ren et al., BMC Cancer 10:27 (2010); Ren et al., J Biomater Sci Polym Ed 21:303-314 (2010)); a poly(lactide-co-glycolide) (PLGA) nanoparticle (Cheng et al., Molec Pharm 9:1481-1488 (2012); Babar et al., PNAS e1695-e1704 (2012)), a disialoganglioside-targeting silica nanoparticle or a modified ultrasmall magnetic nanoparticle (Tivnan et al., PLoS One 7:e38129 (2012); Yigit et al., Oncogene 32, 1530-1538 (2013)). In exemplary embodiments, the RNA molecule is part of an extracellular vesicle, such as e.g., an exosome. Such vesicles for delivery of small RNA molecules are described in Hagiwara et al., Drug Deliv and Transl Res 4: 31-37 (2014); Momen-Heravi et al., Nature Scientific Reports 5: 09991 (2015); Wang et al., Asian PAc J Cancer Prev 16(10): 4203-4209 (2015); and International Application Publication No. WO/2014/028763; and include any vesicle in the extracellular space. In exemplary embodiments, the extracellular vesicle is an exosome. In exemplary embodiments, the exosome is 40-100 nm in diameter and are derived from multivescicular endosomes. In exemplary embodiments, the extracellular vesicle is a microvesicle. In exemplary embodiments, the microvesicle is 50-1000 nm in diameter and is generated by budding at the plasma membrane.

RNAs described herein may be formulated with other components to prepare a pharmaceutical composition. Accordingly, in some embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dimethyl sulfoxide (DMSO), dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC) chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glycerol, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, postassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, U K, 2000). Remington's Pharmaceutical Sciences, Sixteenth Edition, E. W. Martin (Mack Publishing Co., Easton, Pa., 1980), discloses RNA components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In other embodiments RNA may be formulated and delivered in liposomes such as described in Geall et al., PNAS 2012 Sep. 4; 109(36):14604-9. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, the RNA compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120) and liposomes which may deliver small molecule drugs such as, but not limited to, DOXIL® from Janssen Biotech, Inc. (Horsham, Pa.).

In some embodiments, the RNA compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104).

In certain embodiments, the RNA composition herein comprises a PEGylated lipid, protein or polymer. In some embodiments, such compositions are also targeted, which can be achieved by chemically conjugating the desired targeting antibody to protein, polymer or lipids used in the formulation of the RNA. Such techniques are known in the art. See e.g., Gose 2016 Biomacromolecules 17:3672-3682 which describes uptake of lipid-antibody functionalized layer-by-layer biopolymer carriers; see also Lu 2011 International Journal of Nanomedicine, 10 Aug. 2011, dx(.)doi(.)org/10.2147/IJN.S22293.

In certain embodiments, the RNA described herein is formulated in saline (See, e.g., Berglund, Vaccine 1994 December; 12(16):1510-4.). In another embodiment, RNA is encapsulated into protein particles with or without lipid complexes (see for example J Am Chem Soc. 2012 May 30; 134(21):8774-7; 2013 Sep. 3; 10(9):3366-74.).

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capabale of buffering at the desired pH such as, for example, phosphate buffers (e.g., PBS), triethanolamine, Tris, bicine, TAPS, tricine, HEPES, HBSS, TES, MOPS, PIPES, cacodylate, IVIES, and others.

U.S. Publ. No. 20150111248, describes compositions and methods for the preparation, manufacture and therapeutic use of polynucleotides, primary transcripts and modified mRNA molecules which have structural and/or chemical features that avoid one or more of the problems known in the art, for example, features which are useful for optimizing formulation and delivery of nucleic acid-based therapeutics while retaining structural and functional integrity, overcoming the threshold of expression, improving expression rates, half-life and/or protein concentrations, optimizing protein localization, and avoiding deleterious bio-responses such as the immune response and/or degradation pathways.

U.S. Publ. No. 20130156849, describes formulation compositions comprising modified nucleic acid molecules which may encode a protein, a protein precursor, or a partially or fully processed form of the protein or a protein precursor.

Other publications which disclose RNA modifications to protect, stabilize or otherwise assist in vivo protein production from RNA or modified RNA include U.S. Publ. No. 20130253178, U.S. Publ. No. 20110143397, U.S. Publ. No. 20060036087, U.S. Publ. No. 20120195936, and U.S. Publ. No. 20100047261.

In other embodiments, commercially available transfection reagents, such as TransMessenger (Qiagen, Venlo, Netherlands), TransIT (Mirus Bio, Madison, Wis., USA), MessengerMax (ThermoFisher Scientific, Waltham, Mass., USA), and Xfect (Clontech, Mountain View, Calif., USA), may be used according to the invention.

As described herein, RNAs of the present disclosure can be formulated with catonic polymers, such as polyethylenimine, including jetPEI (Polyplus, Illkirch, France). Poly(ethylenimine)s (PEIs) were the first materials of the positive polyelectrolyte type to have been introduced into the field of synthetic cationic polymeric vectors by Boussif et al., in 1995 (Proc. Natl. Acad. Sci. USA, 1995, 92, 7297-7301). Two types of PEI can be distinguished based on the macromolecular structure: LPEI with a linear macrostructure (obtained by hydrolysis of poly(2-alkyl-2-oxazoline)), and BPEI with a branched structure (obtained by polymerization of ethylenimine).

In some embodiments, RNA compositions are formulated in a nanoparticle. (See, for example, US Pub. 2018/0008694). In some embodiments, RNA compositions are formulated in a lipid nanoparticle. In some embodiments, the RNA compositions are formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, the RNA compositions are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(omega.-methoxy-poly(ethyleneglycol)2000)carbamoyl)]-1,2-dimyristy-loxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLIin-MC2-DMA, DLin-MC3-DMA, DLIin-MC4-DMA, DLin-DMA, C12-200 and DLin-KC2-DMA.

In some embodiments, the RNA composition comprises a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methy-1}propan-1-ol (Compound 2 in US20130150625); 2-amino-3-[(9Z,12Z)-octadeca-9,12-dien-1-yloxy]-2-[(octyloxy)methyl]propa-n-1-ol (Compound 3 in US20130150625); and 2-(dimethylamino)-3-[(9Z,12Z)-octadeca-9,12-dien-1-yloxy]-2-{[(9Z,12Z)-oc-tadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable lipid, for example, an ionizable cationic lipid such as 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid:5-25% neutral lipid:25-55% sterol; 0.5-15% PEG-lipid.

In some embodiments, lipid nanoparticle formulations consist essentially of a lipid mixture in molar ratios of 20-70% cationic lipid:5-45% neutral lipid:20-55% cholesterol:0.5-15% PEG-modified lipid. In some embodiments, lipid nanoparticle formulations consist essentially of a lipid mixture in a molar ratio of 20-60% cationic lipid:5-25% neutral lipid:25-55% cholesterol:0.5-15% PEG-modified lipid. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578.

In certain embodiments, the lipid nanoparticles for use herein may comprise any one or more of the lipid nanoparticles or any one or more of the lipids as disclosed in US20170283367, WO2017117528, US20170119904, US20160376224, US20160317676, WO2015199952, WO2011075656, WO2016176330, and can be made using the methods described therein.

In certain embodiments, the LNP for use herein include the representative cationic lipids as described and tested in US20170119904. The cationic lipids described in US20170119904 may be formulated and tested using methods known in the art.

The molar ratios of the illustrative cationic lipids and other compounds described in US20170119904 and one or more of the other LNP components as disclosed herein for any given formulation may be modified and optimized using methods known in the art and as described herein. For example, molar ratios of the different components as described herein may range from 40-60% cationic lipid/5-15% neutral lipid/35-45% steroid (e.g., Cholesterol)/0.5-3% PEG lipid.

The size of the LNPs for use herein is generally <100 nm. In certain embodiments, the LNP size ranges from 40-100 nm. In other embodiments, the particles disclosed herein range in size from 45-95 nm, from 50-90 nm, from 55-85 nm, from 60-80 nm, from 65-75 nm.

In one embodiment, a lipid particle for use herein comprises a neutral lipid selected from DSPC, DPPC, POPC, DOPE, or sphingomyelin (SM); a PEG lipid capable of reducing aggregation; and a sterol. In certain embodiments, the lipid particle comprises a first cationic lipid present in a molar ratio of 0% to 60% and a second cationic lipid present in a molar ratio of 0% to 60%, provided that the molar ratio of all cationic lipids in the particle is between about 20% and about 60%; a neutral lipid is present in a molar ratio of about 5% to about 25%; a sterol is present in a molar ratio of about 25% to about 55%; and a PEG lipid is PEG-DMA, PEG-DMG, or a combination thereof, and is present in a molar ratio of about 0.5% to about 15%.

In other embodiments, the lipid nanoparticles for use herein may comprise any one or more of the lipid nanoparticles or any one or more of the lipids as disclosed in WO2008042973, WO2010054406A1; WO2010054384A1; WO2010054401A1; WO2010054405A1, U.S. Pat. Nos. 8,158,601; 9,394,234; WO2011075656A1; WO2009126933A3; WO2002034236A2; WO2010042877A1; U.S. Pat. No. 9,694,077; US20160024498A1; U.S. Pat. No. 9,814,777; WO2013126803A1 WO2006007712, WO2005120152, WO2005026372, WO1996040964.

In certain embodiments, the RNA as described herein is comprised in a formulation that comprise a lipid vesicle formulation comprising: (a) a plurality of lipid vesicles, wherein each lipid vesicle comprises: a cationic lipid; an amphipathic lipid; and a polyethyleneglycol (PEG)-lipid; and (b) RNA (e.g., srRNA), wherein between about 60%-100% of the RNA in the formulation is fully encapsulated in the lipid vesicles. In this regard, in some embodiments, at least 60%, 65%, 66%, 67%, 68%, 69%, 70%, 75%, 80%, 85%, 90%, 95%, 96%, 97%, 98%, or 99% of the RNA in the formulation is fully encapsulated in the lipid vesicles.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4- to 6-fold lower than the same particles diffusing in water (Lai et al. PNAS 2007 104:1482-487; Lai et al. Adv Drug Deliv Rev. 2009 61: 158-171). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028.

The lipid nanoparticle engineered to penetrate mucus may comprise a polymeric material (i.e. a polymeric core) and/or a polymer-vitamin conjugate and/or a block copolymer (including tri-block co-polymers). The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804. The polymeric material may additionally be irradiated. As a non-limiting example, the polymeric material may be gamma irradiated (see e.g., International App. No. WO201282165). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476), and (poly(ethylene glycol))-(poly(propylene oxide))-(poly(ethylene glycol)) triblock copolymer (see e.g., U.S. Publication 20120121718 and U.S. Publication 20100003337 and U.S. Pat. No. 8,263,665). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600). A non-limiting scalable method to produce nanoparticles which can penetrate human mucus is described by Xu et al. (see, e.g., J Control Release 2013, 170:279-86).

In some embodiments, the RNA composition is formulated as a solid lipid nanoparticle. A solid lipid nanoparticle (SLN) may be spherical with an average diameter between 10 to 1000 nm. SLNs possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2, pp 1696-1702). As a non-limiting example, the SLN may be the SLN described in International Patent Publication No. WO2013105101. As another non-limiting example, the SLN may be made by the methods or processes described in International Patent Publication No. WO2013105101.

In some embodiments, the RNA may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.). In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA composition comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052.

As a non-limiting example, the therapeutic nanoparticle comprises a PLGA-PEG block copolymer (see U.S. Publication No. US20120004293 and U.S. Pat. No. 8,236,330). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923). In yet another non-limiting example, the therapeutic nanoparticle is a stealth nanoparticle or a target-specific stealth nanoparticle as described in U.S. Patent Publication No. US20130172406.

In some embodiments, the RNA may be encapsulated in, linked to and/or associated with zwitterionic lipids. Non-limiting examples of zwitterionic lipids and methods of using zwitterionic lipids are described in U.S. Patent Publication No. US20130216607. In some aspects, the zwitterionic lipids may be used in the liposomes and lipid nanoparticles described herein.

In some embodiments, the RNA compositions may be formulated in colloid nanocarriers as described in U.S. Patent Publication No. US20130197100.

In other embodiments, the RNA compositions may be formulated using synthetic nanocarriers such as those described in WO2014153114 and WO2017181110.

In certain embodiments, the RNA may be encapsulated in a gel such as described in Blackburn 2009 Bioconjug Chem. 20(5):960-968; Knipe 2016 Biomacromolecules, 17, 788-797.

In some embodiments, the nanoparticles described herein may be optimized for oral administration. The nanoparticle may comprise at least one cationic biopolymer such as, but not limited to, chitosan or a derivative thereof. As a non-limiting example, the nanoparticle may be formulated by the methods described in U.S. Publication No. 20120282343.

In some embodiments of the invention, lipid nanoparticles described in the art as useful in the fields of siRNA, miRNA and RNAi are contemplated herein. For example, the lipid particles, including the various components and molar ratios and physical properties, described in 20170143631 are contemplated for use herein. A lipid particle can include one or more of a second amino lipid or cationic lipid, a neutral lipid, a sterol, and a lipid selected to reduce aggregation of lipid particles during formation, which may result from steric stabilization of particles which prevents charge-induced aggregation during formation.

As used herein, the term “cationic lipid” is meant to include those lipids having one or two fatty acid or fatty alkyl chains and an amino head group (including an alkylamino or dialkylamino group) that may be protonated to form a cationic lipid at physiological pH. In some embodiments, a cationic lipid is referred to as an “amino lipid.” Other cationic lipids would include those having alternative fatty acid groups and other dialkylamino groups, including those in which the alkyl substituents are different (e.g., N-ethyl-N-methylamino-, N-propyl-N-ethylamino- and the like). In general, lipids (e.g., a cationic lipid) having less saturated acyl chains are more easily sized, particularly when the complexes are sized below about 0.3 microns, for purposes of filter sterilization. Cationic lipids containing unsaturated fatty acids with carbon chain lengths in the range of C10 to C20 are preferred. Other scaffolds can also be used to separate the amino group (e.g., the amino group of the cationic lipid) and the fatty acid or fatty alkyl portion of the cationic lipid. Suitable scaffolds are known to those of skill in the art.

In certain embodiments, cationic lipids have at least one protonatable or deprotonatable group, such that the lipid is positively charged at a pH at or below physiological pH (e.g. pH 7.4), and neutral at a second pH, preferably at or above physiological pH. Such lipids are also referred to as cationic lipids. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwiterrionic, are not excluded from use in the invention.

In certain embodiments, protonatable lipids (i.e., cationic lipids) have a pKa of the protonatable group in the range of about 4 to about 11. Most preferred is pKa of about 4 to about 7, because these lipids will be cationic at a lower pH formulation stage, while particles will be largely (though not completely) surface neutralized at physiological pH around pH 7.4. One of the benefits of this pKa is that at least some nucleic acid associated with the outside surface of the particle will lose its electrostatic interaction at physiological pH and be removed by simple dialysis; thus greatly reducing the particle's susceptibility to clearance.

Examples of lipids that reduce aggregation of particles during formation include polyethylene glycol (PEG)-modified lipids, monosialoganglioside Gm1, and polyamide oligomers (“PAO”) such as (described in U.S. Pat. No. 6,320,017). Other compounds with uncharged, hydrophilic, steric-barrier moieties, which prevent aggregation during formulation, like PEG, Gm1 or ATTA, can also be coupled to lipids for use as in the methods and compositions of the invention. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids).

Examples of lipids that reduce aggregation and/or are suitable for conjugation to nucleic acid agents that can be used in the liver screening model are polyethylene glycol (PEG)-modified lipids, monosialoganglioside Gm1, and polyamide oligomers (“PAO”) such as (described in U.S. Pat. No. 6,320,017). Other compounds with uncharged, hydrophilic, steric-barrier moieties, which prevent aggregation during formulation, like PEG, Gm1 or ATTA, can also be coupled to lipids for use as in the methods and compositions of the invention. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids).

Specific examples of PEG-modified lipids (or lipid-polyoxyethylene conjugates) that are useful in the invention can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873 PEG-modified dialkylamines and PEG-modified 1,2-diacyloxypropan-3-amines. Particularly preferred are PEG-modified diacylglycerols and dialkylglycerols. In some embodiments, the total mol % of PEG lipids within a particle is about 1.5 mol %. For example, when the particle includes a plurality of PEG lipids described herein such as a PEG-modified lipid as described above and a targeting lipid containing a PEG, the total amount of the PEG containing lipids when taken together is about 1.5 mol %.

In embodiments where a sterically-large moiety such as PEG or ATTA are conjugated to a lipid anchor, the selection of the lipid anchor depends on what type of association the conjugate is to have with the lipid particle. It is well known that mePEG (mw2000)-diastearoylphosphatidylethanolamine (PEG-DSPE) will remain associated with a liposome until the particle is cleared from the circulation, possibly a matter of days. Other conjugates, such as PEG-CerC20 have similar staying capacity. PEG-CerC14, however, rapidly exchanges out of the formulation upon exposure to serum, with a T_{1/2} less than 60 mins. in some assays. As illustrated in U.S. Pat. No. 5,820,873, at least three characteristics influence the rate of exchange: length of acyl chain, saturation of acyl chain, and size of the steric-barrier head group. Compounds having suitable variations of these features may be useful for the invention. For some therapeutic applications, it may be preferable for the PEG-modified lipid to be rapidly lost from the nucleic acid-lipid particle in vivo and hence the PEG-modified lipid will possess relatively short lipid anchors. In other therapeutic applications, it may be preferable for the nucleic acid-lipid particle to exhibit a longer plasma circulation lifetime and hence the PEG-modified lipid will possess relatively longer lipid anchors. Exemplary lipid anchors include those having lengths of from about C14 to about C22, preferably from about C14 to about C16. In some embodiments, a PEG moiety, for example an mPEG-NH₂, has a size of about 1000, 2000, 5000, 10,000, 15,000 or 20,000 daltons.

It should be noted that aggregation preventing compounds do not necessarily require lipid conjugation to function properly. Free PEG or free ATTA in solution may be sufficient to prevent aggregation. If the particles are stable after formulation, the PEG or ATTA can be dialyzed away before administration to a subject.

Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C14 to C22 are preferred. In another group of embodiments, lipids with mono or diunsaturated fatty acids with carbon chain lengths in the range of C14 to C22 are used. Additionally, lipids having mixtures of saturated and unsaturated fatty acid chains can be used. Preferably, the neutral lipids used in the invention are DOPE, DSPC, DPPC, POPC, or any related phosphatidylcholine. The neutral lipids useful in the invention may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Other cationic lipids, which carry a net positive charge at about physiological pH, in addition to those specifically described above, may also be included in lipid particles of the invention. Such cationic lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.C1”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available froth GIBCO/BRL). In particular embodiments, a cationic lipid is an amino lipid.

Anionic lipids suitable for use in lipid particles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In numerous embodiments, amphipathic lipids are included in lipid particles of the invention. “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine (DPPC), dioleoylphosphatidylcholine (DOPC), distearoylphosphatidylcholine (DSPC), dimyristoylphosphatidyl choline (DMPC), or dilinoleylphosphatidylcholine (DLPC). Other phosphorus-lacking compounds, such as sphingolipids, glycosphingolipid families, diacylglycerols, and.beta.-acyloxyacids, can also be used. Additionally, such amphipathic lipids can be readily mixed with other lipids, such as triglycerides and sterols.

Also suitable for inclusion in the lipid particles of the invention are programmable fusion lipids. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid particle membrane over time. Exemplary lipid anchors include those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons.

By the time the lipid particle is suitably distributed in the body, it has lost sufficient cloaking agent so as to be fusogenic. With other signal events, it is desirable to choose a signal that is associated with the disease site or target cell, such as increased temperature at a site of inflammation.

A lipid particle conjugated to a nucleic acid agent can also include a targeting moiety, e.g., a targeting moiety that is specific to a cell type or tissue. Targeting of lipid particles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). Exemplary targeting moieties include a targeting lipid such as a targeting lipid described herein. In some embodiments, the targeting lipid is a GalNAc containing targeting lipid such as GalNAc3-DSG and GalNAc3-PEG-DSG as described herein. The targeting moieties can include the entire protein or fragments thereof. Targeting mechanisms generally require that the targeting agents be positioned on the surface of the lipid particle in such a manner that the targeting moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fl. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

In various embodiments, the RNA is formulated in a stable emulsion. Oil-in-water emulsions are known for adjuvanting vaccines. RNA delivery according to the present invention can utilize an oil-in-water emulsion, provided that the emulsion includes one or more cationic molecules. For instance, a cationic lipid can be included in the emulsion to provide a positive droplet surface to which negatively-charged RNA can attach.

The emulsion comprises one or more oils. Suitable oil(s) include those from, for example, an animal (such as fish) or a vegetable source. The oil is ideally biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Illustrative emulsions comprise squalene, a shark liver oil which is a branched, unsaturated terpenoid (C30H50; [(CH₃)₂C[.dbd.CHCH₂CH₂C(CH₃)]₂.dbd.CHCH₂—]2; 2,6,10,15,19,23-hexamethyl-2,6,10,14,18,22-tetracosahexaene; CAS RN 7683-64-9). Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the alpha, beta, gamma, delta, epsilon or xi tocopherols can be used, but alpha-tocopherols are preferred. D-alpha-tocopherol and DL-alpha-tocopherol can both be used. A preferred alpha-tocopherol is DL-alpha-tocopherol. An oil combination comprising squalene and a tocopherol (e.g. DL-alpha-tocopherol) can be used. The oil in the emulsion may comprise a combination of oils e.g. squalene and at least one further oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. Preferably this lipid is a surfactant so that it can facilitate formation and stabilization of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′-[N—(N′,N′-Dimethylaminoethane)-carbamoyl]Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimethylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate, N-alkyl pyridinium salts (e.g. cetylpyridinium bromide and cetylpyridinium chloride), N-alkylpiperidinium salts, dicationic bolaform electrolytes (C₁₂Me₆; C₁₂Bu₆), dialkylglycetylphosphorylcholine, lysolecithin, L-alpha. dioleoyl-phosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (C₁₂GluPhCₙN⁺), cationic derivatives of cholesterol, including but not limited to cholesteryl-3.beta.-oxysuccinamidoethylenetrimethylammonium salt, cholesteryl-3. beta.-oxysuccinamidoethylene-dimethylamine, cholesteryl-3.beta.-carboxyamidoethylenetrimethylammonium salt, and cholesteryl-3 beta-carboxyamidoethylenedimethylamine. The cationic lipid is preferably biodegradable (metabolizable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, preferably 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and preferably at least 90%. Apparatuses for determining the average droplet size in an emulsion, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidization. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidization can be performed until an emulsion with a desired droplet size average and distribution are achieved.

As an alternative to microfluidization, thermal methods can be used to cause phase inversion. These methods can also provide a submicron emulsion with a tight particle size distribution.

Preferred emulsions can be filter sterilized i.e. their droplets can pass through a 220 nm filter. As well as providing a sterilization, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml, from about 0.6 mg/ml to about 25 mg/ml, from about 0.7 mg/ml to about 25 mg/ml, from about 0.8 mg/ml to about 25 mg/ml, from about 0.9 mg/ml to about 25 mg/ml, from about 1.0 mg/ml to about 25 mg/ml, from about 1.1 mg/ml to about 25 mg/ml, from about 1.2 mg/ml to about 25 mg/ml, from about 1.3 mg/ml to about 25 mg/ml, from about 1.4 mg/ml to about 25 mg/ml, from about 1.5 mg/ml to about 25 mg/ml, from about 1.6 mg/ml to about 25 mg/ml, from about 1.7 mg/ml to about 25 mg/ml, from about 0.5 mg/ml to about 24 mg/ml, from about 0.5 mg/ml to about 22 mg/ml, from about 0.5 mg/ml to about 20 mg/ml, from about 0.5 mg/ml to about 18 mg/ml, from about 0.5 mg/ml to about 15 mg/ml, from about 0.5 mg/ml to about 12 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1.9 mg/ml, from about 0.5 mg/ml to about 1.8 mg/ml, from about 0.5 mg/ml to about 1.7 mg/ml, from about 0.5 mg/ml to about 1.6 mg/ml, from about 0.6 mg/ml to about 1.6 mg/ml, from about 0.7 mg/ml to about 1.6 mg/ml, from about 0.8 mg/ml to about 1.6 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 12 mg/ml, about 18 mg/ml, about 20 mg/ml, about 21.8 mg/ml, about 24 mg/ml, etc. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.62 mg/ml to about 5 mg/ml, from about 1 mg/ml to about 5 mg/ml, from about 1.5 mg/ml to about 5 mg/ml, from about 2 mg/ml to about 5 mg/ml, from about 2.46 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, from about 0.1 mg/ml to about 4.92 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.46 mg/ml, from about 0.1 mg/ml to about 2 mg/ml, from about 0.1 mg/ml to about 1.5 mg/ml, from about 0.1 mg/ml to about 1 mg/ml, from about 0.1 mg/ml to about 0.62 mg/ml, about 0.15 mg/ml, about 0.3 mg/ml, about 0.6 mg/ml, about 0.62 mg/ml, about 0.9 mg/ml, about 1.2 mg/ml, about 2.46 mg/ml, about 4.92 mg/ml, etc. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 5 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.5 mg/ml, from about 0.1 mg/ml to about 2 mg/ml, from about 0.1 mg/ml to about 1.5 mg/ml, from about 0.1 mg/ml to about 1.45 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.6 mg/ml to about 5 mg/ml, from about 0.73 mg/ml to about 5 mg/ml, from about 0.8 mg/ml to about 5 mg/ml, from about 0.9 mg/ml to about 5 mg/ml, from about 1.0 mg/ml to about 5 mg/ml, from about 1.2 mg/ml to about 5 mg/ml, from about 1.45 mg/ml to about 5 mg/ml, from about 2 mg/ml to about 5 mg/ml, from about 2.5 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, about 1.2 mg/ml, about 1.45 mg/ml, etc. Alternatively, the cationic oil-in-water emulsion may comprise DDA at about 20 mg/ml, about 21 mg/ml, about 21.5 mg/ml, about 21.6 mg/ml, about 25 mg/ml. In an exemplary embodiment, the cationic oil-in-water emulsion comprises from about 0.73 mg/ml to about 1.45 mg/ml DDA, such as 1.45 mg/ml.

Certain preferred compositions of the invention for administration to a patient comprise squalene, span 85, polysorbate 80, and DOTAP. For instance: squalene may be present at 5-15 mg/ml; span 85 may be present at 0.5-2 mg/ml; polysorbate 80 may be present at 0.5-2 mg/ml; and DOTAP may be present at 0.1-10 mg/ml. The emulsion can include the same amount (by volume) of span 85 and polysorbate 80. The emulsion can include more squalene than surfactant. The emulsion can include more squalene than DOTAP.

The ratio of the RNA to the cationic or polycationic compound in the formulation may be calculated on the basis of the nitrogen/phosphate ratio (N/P-ratio) of the entire RNA complex, i.e. the ratio of positively charged (nitrogen) atoms of the cationic or polycationic compound to the negatively charged phosphate atoms of the nucleic acids. In the context of the present disclosure, an N/P-ratio is in the range of about 0.1-40. In certain embodiments, an N/P-ratio is in a range of about 0.5-36, 1-36, 5-36, 8-36, 10-40, 15-40, 8-20, 0.3-9, in a range of about 0.5-8, 0.7-7, 2-8, 3-10, 3-9, 3-8, 4-10, 4-9, 4-8, 4-7, 5-10, 5-9, 5-8, 5-7, 6-10, 6-9, 6-8, 6-7, 7-12, 7-11, 7-10, 7-9, 7-8. In certain embodiments, the N/P ratio is about 1, 1.5, 2, 2.5, 3, 3.5, 4, 4.5, 5, 5.5, 6, 6.5, 7, 7.5, 8, 8.5, 9, 9.5, 10, 10.5, 11, 11.5, or about 12.

In one example, 1 pg of RNA typically contains about 3 nmol phosphate residues, provided the RNA exhibits a statistical distribution of bases. Additionally, 1 pg of peptide typically contains about x nmol nitrogen residues, dependent on the molecular weight and the number of basic amino acids. When calculated for (Arg)g (molecular weight 1424 g/mol, 9 nitrogen atoms), 1 pg (Arg) a contains about 700 pmol (Arg)g and thus 700×B=B300 pmol basic amino acids=FJ.3 nmol nitrogen atoms. For a mass ratio of about 1:1 RNA/(Arg)9 an N/P ratio of about 2 can be calculated. When exemplarily calculated for protamine (molecular weight about 4250 g/mol, 21 nitrogen atoms, when protamine from salmon is used) with a mass ratio of about 2:1 with 2 pg RNA, B nmol phosphate are to be calculated for the RNA; 1 pg protamine contains about 235 pmol protamine molecules and thus 235×21=4935 pmol basic nitrogen atoms=4.9 nmol nitrogen atoms. For a mass ratio of about 2:1 RNA/protamine an N/P ratio of about 0.81 can be calculated. For a mass ratio of about 8:1 RNA/protamine an N/P ratio of about 0.2 can be calculated.

In certain embodiments, the RNA is formulated such that the charge ratio of positive charges to negative charges in the nanoparticles is in a desired range, such as described in WO2013/143683. In this regard, in certain embodiments, the RNA is formulated such that the charge ratio of positive charges to negative charges in the nanoparticles is between 1.4:1 and 1:8, between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In one embodiment, the zeta potential of the nanoparticles is −5 or less, −10 or less, −15 or less, −20 or less or −25 or less. In various embodiments, the zeta potential of the nanoparticles is −35 or higher, −30 or higher or −25 or higher. In one embodiment, the nanoparticles have a zeta potential from 0 mV to −50 mV, preferably 0 mV to −40 mV or −10 mV to −30 mV.

Specifically, zeta potential is a scientific term for electrokinetic potential in colloidal systems. From a theoretical viewpoint, zeta potential is the electric potential in the interfacial double layer at the location of the slipping plane versus a point in the bulk fluid away from the interface. In other words, zeta potential is the potential difference between the dispersion medium and the stationary layer of fluid attached to the dispersed particle. Zeta potential is widely used for quantification of the magnitude of the electrical charge at the double layer.

Zeta potential can be calculated using theoretical models and experimentally-determined electrophoretic mobility or dynamic electrophoretic mobility measurements. Electrokinetic phenomena and electroacoustic phenomena are the usual sources of data for calculation of zeta potential.

Electrophoresis may be used for estimating zeta potential of particulates. In practice, the zeta potential of a dispersion can be measured by applying an electric field across the dispersion. Particles within the dispersion with a zeta potential will migrate toward the electrode of opposite charge with a velocity proportional to the magnitude of the zeta potential. This velocity may be measured using the technique of the Laser Doppler Anemometer. The frequency shift or phase shift of an incident laser beam caused by these moving particles may be measured as the particle mobility, and this mobility may be converted to the zeta potential by inputting the dispersant viscosity and dielectric permittivity, and the application of the Smoluchowski theories.

Electrophoretic velocity is proportional to electrophoretic mobility, which is the measurable parameter. There are several theories that link electrophoretic mobility with zeta potential.

Suitable systems such as the Nicomp 380 ZLS system can be used for determining the zeta potential. Such systems usually measure the electrophoretic mobility and stability of charged particles in liquid suspension. These values are a predictor of the repulsive forces being exerted by the particles in suspension and are directly related to the stability of the colloidal system. A zeta potential may be measured according to a protocol as described below.

Electric charge is a physical property that causes a matter to experience a force when near other electrically charged matter. Electric charge comes in two types, called positive and negative. Charged particles whose charges have the same sign repel one another, and particles whose charges have different signs attract.

The electric charge of a macroscopic object such as a particle is the sum of the electric charges of the particles that make it up. The nanoparticles described herein may have equal numbers of positive and negative charges, in which case their charges cancel out, yielding a net charge of zero, thus making the nanoparticles neutral. Net charge is the charge on a whole object such as a compound.

An ion having an overall net positive charge is a cation while an ion having an overall net negative charge is an anion.

Nanoparticles described herein can be formed by adjusting a positive to negative charge, depending on the (+/−) charge ratio of the cationic lipid to the RNA and mixing the RNA and the cationic lipid. The +/−charge ratio of the cationic lipid to the RNA in the nanoparticles described herein can be calculated by the following equation. (+/−charge ratio)=[(cationic lipid amount (mol))*(the total number of positive charges in the cationic lipid)]:[(RNA amount (mol))*(the total number of negative charges in RNA)]. The RNA amount and the cationic lipid amount can be easily determined by one skilled in the art in view of a loading amount upon preparation of the nanoparticles.

According to an embodiment, the ratio of positive to negative charge in nanoparticles suitable for the invention is such that they may have a global negative charge or a global charge at or near the neutrality.

If the present disclosure refers to a charge such as a positive charge, negative charge or neutral charge or a cationic compound, negative compound or neutral compound this generally means that the charge mentioned is present at a selected pH, such as a physiological pH. For example, the term “cationic lipid” means a lipid having a net positive charge at a selected pH, such as a physiological pH. The term “neutral lipid” means a lipid having no net positive or negative charge and can be present in the form of a non-charge or a neutral amphoteric ion at a selected pH, such as a physiological pH. By “physiological pH” herein is meant a pH of about 7.5.

Additional means to deliver RNA compositions according to various embodiments of the invention include in situ electroporation using, for example, AgilePulse (BTX, Harvard Apparatus, Holliston, Mass., USA). In another embodiment, a gene gun or biolistic gene transfer system may be used (See, e.g., J Clin Diagn Res., 2015, 9(1): GE01-GE06).

Viral vector-based delivery methods are also contemplated in various embodiments. Non-limiting examples include lentiviral vectors as described herein, alphavirus vectors, and adenovirus vectors. Intracellular bacterial delivery of the RNA compositions described herein is also contemplated in various embodiments. (See, e.g., Cellular Microbiol., 2005, 7(5):709-724)

In various embodiments, all of the RNA composition described herein may optionally be formulated with a PAMP such as GLA.

In some embodiments, the effective amount is a total dose of 25 μg to 1000 g, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the effective amount of an RNA (e.g., mRNA or srRNA) is a dose of between about 1 ng to about 1000 ag. In other embodiments, the dose of RNA is between 50-1000 ag. In some embodiments, the effective amount of a RNA (e.g., mRNA or srRNA) is a dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of an RNA (e.g., srRNA) is a dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of a RNA (e.g., srRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of a RNA (e.g., mRNA) vaccine is a total dose of 25, 50, 100, 150, 200, 250, 300, 350, 400, 450, or 500 μg administered to the subject a total of two times.

In certain embodiments, in some cases of intradermal injection, which may be carried out by using a conventional needle, the amount of such RNA construct comprised in a single dose is typically at least 200 μg, from 200 pg to 1,000 pg, from 300 pg to 850 pg, or from 300 pg to 700 pg. In the case of intradermal injection, which can be carried out using devices known in the art (e.g. using a Tropis device; PharmaJet Inc, Boulder Colo., US), the amount of the such RNA construct comprised in a single dose is typically at least 80 pg, from 80 pg to 700 pg, from 80 pg to 400 pg. Moreover, in the case of intramuscular injection, which can be carried out by using a conventional needle or via jet injection, the amount of the such RNA construct comprised in a single dose is typically at least 80 pg, from 80 pg to 1,000 pg, from 80 pg to 850 pg, or from 80 pg to 700 pg. In certain embodiments, the RNA described herein is injected intratumorally. In this regard, an amount of RNA is injected sufficient to express the desired amount of protein encoded by the RNA.

Sequences of Interest (SOI) Encoded by the RNA

The RNAs described herein encode an immunomodulatory molecule. In certain embodiments, the RNAs described herein encode IL-12 and, optionally, other sequences of interest including, but not limited to, other immunomodulatory molecules, cytokines, chemokines, antigens of interest, checkpoint inhibitors, etc.

Polynucleotide sequences encoding IL-12 are known in the art and available in public databases. As would be readily understood by the person of ordinary skill in the art, IL-12 is a heterodimeric cytokine with multiple biological effects on the immune system. It is composed of two subunits, p35 and p40, both of which are required for the secretion of the active form of IL-12, p70. In one embodiment the IL-12 sequence (expression cassette) comprises a polynucleotide that directs expression of IL-12 polypeptide. Any IL-12 polypeptide including variants and derivatives of known IL-12 molecules can be used, where variants and derivatives retain IL-12 activity. IL-12 activity can be measured using assays known in the art (e.g., such as described in Example 1). In one embodiment, the IL-12 is human IL-12. In another embodiment, the IL-12 is murine IL-12. In one embodiment the polynucleotide comprises the sequence of both IL-12 subunits, p35 and p40, separated by an IRES sequence which permits expression of multiple transgenes from a single transcript. In particular embodiments the vectors described herein encode a single chain IL-12 (scIL-12). In this regard the single chain fusion protein may encode IL-12 subunits in either orientation, and in certain embodiments may include a linker between the 2 subunits, such as p35-L-p40 or p40-L-p35. A “linker” is a peptide that joins or links other peptides or polypeptides, such as a linker of about 2 to about 150 amino acids. Any of a variety of linkers are known in the art and can be used herein (see e.g., Adv Drug Deliv Rev. 2013 October; 65(10):1357-69). In certain embodiments, the linker is an elastin linker.

Other sequences of interest may also be encoded by the RNAs described herein. The RNAs described herein optionally comprise one or more sequences of interest. The RNAs herein comprise a sequence of interest which encodes a protein of interest or otherwise encodes a functional RNA (miRNA, siRNA, tRNA, etc).

In certain configurations, the sequence of interest encodes an immunomodulatory molecule. Exemplary immunomodulatory molecules include any of a variety of cytokines. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1 through IL-36, including, IL-1, IL-1alpha, IL-2, IL-3, IL-4, IL-5, IL-6, IL-7, IL-8, IL-9, IL-10, IL-11, IL-12; IL-15, IL-18, IL-21, IL-23, IL-27, TNF; and other polypeptide factors including LIF and kit ligand (KL). Other immunomodulatory molecules contemplated for use herein include Stimulator of interferon genes (STING) (also referred to as TMEM173 or MPYS/MITA/ERISIRF3), B7.1, B7.2, 4-1BB, CD40 ligand (CD40L), drug-inducible CD40 (iCD40), and the like.

In certain cases, the sequence of interest can be a gene encoding a short interfering RNA (siRNA) or a microRNA (miRNA) of interest that down-regulates expression of a molecule. For example, the gene encoding an siRNA or a microRNA can be used to down-regulate expression of negative regulators in a cell, including those that inhibit activation or maturation of dendritic cells. siRNAs and microRNAs are well known in the art (Fire et al., Nature 391:806, 1998; see also “The RNA Interference Resource” of Applied Biosystems, Trang et al., Oncogene Suppl 2:S52, 2008; Taganov, K., et al. 2007. Immunity 26:133-137; Dahlberg, J. E. and E. Lund. 2007. Sci. STKE 387:pe25; Tiemann and Rossi, EMBO Mol Med 1:142, 2009). Alternatively, the sequence of interest can encode a self-complementary double stranded RNA in which the complementary region is greater than about 20 ribonucleotides in length, or an anti-sense RNA that is greater than about 20 ribonucleotides in length. Those of ordinary skill in the art will appreciate that siRNA, miRNA, dsRNA and anti-sense RNA molecules can be expressed from an RNA polymerase III promoter, or, alternatively, can be a component of a non-coding RNA that is transcribed from an RNA polymerase II promoter.

In certain embodiments, the immunomodulatory molecule encoded by the SOI described herein is a checkpoint inhibitor molecule. Immune checkpoints refer to a variety of inhibitory pathways of the immune system that are crucial for maintaining self-tolerance and for modulating the duration and amplitude of an immune responses. Tumors use certain immune-checkpoint pathways as a major mechanism of immune resistance, particularly against T cells that are specific for tumor antigens. (see., e.g., Pardoll, 2012 Nature 12:252; Chen and Mellman 2013 Immunity 39:1). The present disclosure provides nucleic acid sequences of interest encoding immune checkpoint inhibitors. Immune checkpoint inhibitors include any agent that blocks or inhibits in a statistically significant manner, the inhibitory pathways of the immune system. Such inhibitors may include antibodies, or antigen binding fragments thereof, that bind to and block or inhibit immune checkpoint receptors or antibodies that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55) and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), Pembrolizumab (MK-3475; PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

In another embodiment, the sequence of interest encodes a “binding domain” or “binding region” or “binding element”. According to the present disclosure, binding domains may be, for example, any protein, polypeptide, oligopeptide, or peptide that possesses the ability to specifically recognize and bind to a biological molecule (e.g., a cell surface receptor or tumor associated protein, or a component thereof). A binding domain includes any naturally occurring, synthetic, semi-synthetic, or recombinantly produced binding partner for a biological molecule of interest. For example, a binding domain may be antibody light chain and heavy chain variable regions, or the light and heavy chain variable region regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE™ analysis). In certain embodiments, the RNA herein may contain an SOI that encodes a binding domain that is an agonist for a cytokine receptor, such as the IL12 binding receptor.

In certain embodiments, the target molecule to be bound by a binding domain may be a cell surface expressed protein, such as a receptor (e.g., immune checkpoint molecule) or a tumor antigen. In another embodiment, the target molecule bound by a binding domain useful herein is a soluble antigen such as a cytokine, albumin, or other serum protein. Illustrative binding domains include immunoglobulin antigen-binding domains such as scFv, scTCR, extracellular domains of receptors, ligands for cell surface molecules/receptors, or receptor binding domains thereof, and tumor binding proteins. In certain embodiments, the antigen binding domains can be an scFv, a VH, a VL, a domain antibody variant (dAb), a camelid antibody (VHH), a fibronectin 3 domain variant, an ankyrin repeat variant and other antigen-specific binding domain derived from other protein scaffolds.

A sequence encoding a detectable product, usually a protein, can be included to allow for identification of cells that are expressing the desired product. For example, a fluorescent marker protein, such as green fluorescent protein (GFP), is incorporated into the construct along with a sequence of interest (e.g., encoding an antigen). In other cases, the protein may be detectable by an antibody or the protein may be an enzyme that acts on a substrate to yield a detectable product, or a product that allows selection of a transfected or transduced target cell, for example confers drug resistance, such as hygromycin resistance. Typical selection genes encode proteins that confer resistance to antibiotics or other toxins suitable for use in eukaryotic cells, e.g., neomycin, methotrexate, blasticidine, among others known in the art, or complement auxotrophic deficiencies, or supply critical nutrients withheld from the media. The selectable marker can optionally be present on a separate plasmid and introduced by co-transfection.

In certain embodiments, the SOI for use herein may comprise a sequence encoding a fusion protein. In such sequences of interest encoding a fusion protein, the various elements to be expressed are operably linked to be expressed in a functional manner. In some configurations, a fusion sequence comprises a sequence of interest and a sequence encoding a reporter product. In other embodiments, such as IL12, multi chain proteins are expressed with a linker connecting them. In certain embodiments, the linker or other portions of the expressed protein, may be cleavable via a cleavage sequence.

Any of the sequences of interest described herein may comprise a variant of the wild type version of the sequence of interest, either of the nucleotide sequence or the amino acid sequence, or both.

With respect to nucleic acid sequences (e.g., RNA sequences, etc), polynucleotide variants may contain one or more substitutions, additions, deletions and/or insertions, such that the biological function of the polypeptide encoded by the variant polynucleotide is not substantially diminished relative to the reference polypeptide encoded by the polynucleotide sequence. In one non-limiting example, certain sequences of interest may include a sequence encoding an immunomodulatory molecule such as IL-12. A variant of a polynucleotide sequence encoding an immunomodulatory molecule may comprise one or more substitutions, additions, deletions and/or insertions, preferably such that the immunomodulatory activity of the molecule encoded thereby is not substantially diminished relative to the reference immunomodulatory molecule encoded by the polynucleotide sequence. In this regard, such polynucleotides encode polypeptides that have a level of biological activity (e.g., immunomodulatory activity) of at least about 50%, 55%, 60%, 65%, 70%, 75%, 80%, 85%, 90%, 95%, 96%, 97%, 98%, or 99% of that for a reference polypeptide sequence. As a non-limiting example, the immunomodulatory protein may be IL-12. IL-12 activity may be measured using assays known to the person of ordinary skill in the art. The activity of a variant IL-12 polypeptide can be measured and compared to a wild-type IL-12 protein.

Fragments of a particular nucleic acid sequence (e.g., RNA, any of the sequences of interest described herein in the form of RNA or incorporated into a vector genome or plasmid for making any of the nucleic acids herein) are also contemplated herein. A fragment of a nucleic acid sequence consists of a continuous stretch of nucleotides corresponding to a continuous stretch of nucleotides in the full-length reference nucleic acid sequence which is the basis for the nucleic acid sequence of the fragment. A nucleic acid (polynucleotide) fragment for use herein may comprise or consist of from about 10% to about 98% of the full-length nucleic acid sequence. In certain embodiments, a fragment nucleic acid comprises or consists of from about 12%-97%, 15%-95%, 20%-90%, 25%-85%, 30%-80%, 35%-75%, 40%-70%, 45%-65%, 50%-80%, 60%-90%, 75%-98%, 80%-95% of the full-length reference nucleic acid. In certain embodiments, a fragment comprises or consists of at least 20 percent, at least 30 percent, at least 40 percent, at least 50 percent, at least 60 percent, at least 70 percent, at least 80 percent, or at least 90, 95, or 99 percent of the full-length nucleic acid sequence. Such a fragment, in the sense of the present invention, is preferably a functional fragment which encodes a protein whose activity is not significantly diminished as compared to the activity of the protein encoded by the full-length nucleic acid. In certain embodiments, “functional fragment” means that the fragment encodes a protein that exhibits substantially the same level of immunogenicity as the protein encoded by the designated nucleic acid comprising the full-length sequence (i.e., the fragment retains a level of immunogenicity to a statistically, clinically, and/or biologically significant degree compared with the immunogenicity of the full-length sequence). In certain embodiments, “functional fragment” means that the fragment encodes a protein that exhibits substantially the same biological activity as the protein encoded by the designated nucleic acid comprising the full-length sequence (i.e., the fragment retains a level of biological activity to a statistically, clinically, and/or biologically significant degree compared with the biological activity of the full-length sequence).

In certain embodiments, the variants may be a codon optimized variant polynucleotide sequence that encodes a protein identical to a reference protein.

### Pharmaceutical Compositions and Kits

Also contemplated herein are pharmaceutical compositions and kits containing PAMP preparations and compositions comprising RNA, and one or more components. Pharmaceutical compositions can include the PAMP or RNA as provided herein and a pharmaceutical carrier. Kits can include the pharmaceutical compositions and/or combinations provided herein, and one or more components, such as instructions for use, vials containing the compositions, a device for mixing the compositions, and/or a device for administering one or more compositions to a subject.

Provided herein are pharmaceutical compositions containing PAMP preparations or RNA as provided herein and a suitable pharmaceutical carrier. Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body.

In addition to the PAMP and/or RNA, the compositions described herein may additionally include and adjuvant. The term “adjuvant” is typically understood not to comprise agents which confer immunity by themselves. An adjuvant assists the immune system nonspecifically to enhance the antigen-specific immune response by e.g. promoting presentation of an antigen to the immune system or induction of an unspecific innate immune response. Furthermore, an adjuvant may preferably e.g. modulate the antigen-specific immune response by e.g. shifting the dominating Th2-based antigen specific response to a more Thl-based antigen specific response or vice versa. Accordingly, an adjuvant may favorably modulate cytokine expression/secretion, antigen presentation, type of immune response etc.

The compositions comprising PAMP preparations and compositions comprising RNA described herein may be administered along with an adjuvant. The adjuvant may be administered with the composition comprising a PAMP or the composition comprising the RNA, before the composition comprising a PAMP or the composition comprising the RNA, or after the composition comprising a PAMP or the composition comprising the RNA. If administered with, the adjuvant may be in the same composition as the PAMP preparation or the composition comprising RNA or may be in a separate composition but administered at the same time. Desirable adjuvants do not significantly disrupt the integrity of the PAMP. Similarly, if administered with the composition comprising RNA, desirable adjuvants do not significantly affect the properties or activity of the formulated RNA.

A variety of adjuvants can be used in combination with the compositions herein to elicit an immune response. Illustrative adjuvants for use herein include glucopyranosyl lipid A (GLA; see e.g., U.S. Pat. Nos. 8,273,361 and 8,609,114; and US published patent application 2015/0335736), alum, 3 De-O-acylated monophosphoryl lipid A (MPL) (see GB 2220211). QS21 is a triterpene glycoside or saponin isolated from the bark of the Quillaja Saponaria Molina tree found in South America (see Kensil et al., in Vaccine Design: The Subunit and Adjuvant Approach (eds. Powell and Newman, Plenum Press, N Y, 1995); U.S. Pat. No. 5,057,540). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Alternatively, Aβ can be coupled to an adjuvant. For example, a lipopeptide version of Aβ can be prepared by coupling palmitic acid or other lipids directly to the N-terminus of Aβ as described for hepatitis B antigen vaccination (Livingston, J. Immunol. 159, 1383-1392 (1997)). However, such coupling should not substantially change the conformation of Aβ so as to affect the nature of the immune response thereto. Adjuvants can be administered as a component of a therapeutic composition with an active agent or can be administered separately, before, concurrently with, or after administration of the active agent.

One class of adjuvants is aluminum salts (alum), such as aluminum hydroxide, aluminum phosphate, aluminum sulfate. Such adjuvants can be used with or without other specific immunostimulating agents such as MPL or 3-DMP, QS21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalane, 0.4% Tween 80, 5% pluronic-blocked polymer L121, and thr-MDP, either microfluidized into a submicron emulsion or vortexed to generate a larger particle size emulsion, and (c) Ribi adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™). Another class of preferred adjuvants is saponin adjuvants, such as Stimulon™ (QS21, Aquila, Worcester, Mass.) or particles generated there from such as ISCOMs (immunostimulating complexes) and ISCOMATRIX. Other adjuvants include Complete Freund's Adjuvant (CFA) and Incomplete Freund's Adjuvant (IFA). Other adjuvants include cytokines, such as interleukins (IL-1, IL-2, and IL-12), macrophage colony stimulating factor (M-CSF), tumor necrosis factor (TNF).

In some embodiments, the adjuvant may be a lipid nanoparticle. In certain embodiments, the lipid nanoparticles for use herein may comprise any one or more of the lipid nanoparticles or any one or more of the lipids as disclosed in US20170283367, WO2017117528, US20170119904, US20160376224, US20160317676, WO2015199952, WO2011075656, WO2016176330, and can be made using the methods described therein.

In certain embodiments, the LNP for use herein include the representative cationic lipids as described and tested in US20170119904. The cationic lipids described in US20170119904 may be formulated and tested using methods known in the art.

The molar ratios of the illustrative cationic lipids and other compounds described in US20170119904 and one or more of the other LNP components as disclosed herein for any given formulation may be modified and optimized using methods known in the art and as described herein. For example, molar ratios of the different components as described herein may range from 40-60% cationic lipid/5-15% neutral lipid/35-45% steroid (e.g., Cholesterol)/0.5-3% PEG lipid.

The size of the LNPs for use herein is generally <100 nm. In certain embodiments, the LNP size ranges from 40-100 nm. In other embodiments, the particles disclosed herein range in size from 45-95 nm, from 50-90 nm, from 55-85 nm, from 60-80 nm, from 65-75 nm.

In one embodiment, a lipid particle for use herein comprises a neutral lipid selected from DSPC, DPPC, POPC, DOPE, or sphingomyelin (SM); a PEG lipid capable of reducing aggregation; and a sterol. In certain embodiments, the lipid particle comprises a first cationic lipid present in a molar ratio of 0% to 60% and a second cationic lipid present in a molar ratio of 0% to 60%, provided that the molar ratio of all cationic lipids in the particle is between about 20% and about 60%; a neutral lipid is present in a molar ratio of about 5% to about 25%; a sterol is present in a molar ratio of about 25% to about 55%; and a PEG lipid is PEG-DMA, PEG-DMG, or a combination thereof, and is present in a molar ratio of about 0.5% to about 15%.

In other embodiments, the lipid nanoparticles for use herein may comprise any one or more of the lipid nanoparticles or any one or more of the lipids as disclosed in WO2008042973, WO2010054406A1; WO2010054384A1; WO2010054401A1; WO2010054405A1, U.S. Pat. Nos. 8,158,601; 9,394,234; WO2011075656A1; WO2009126933A3; WO2002034236A2; WO2010042877A1; U.S. Pat. No. 9,694,077; US20160024498A1; U.S. Pat. No. 9,814,777; WO2013126803A1 WO2006007712, WO2005120152, WO2005026372, WO1996040964.

An adjuvant can be administered with the compositions comprising PAMPs of the invention as a single composition, or can be administered before, concurrent with or after administration of the composition comprising a PAMP. An adjuvant can be administered with the compositions comprising RNA as a single composition, or can be administered before, concurrent with or after administration of such RNA compositions. Any of the compositions disclosed herein (e.g., comprising PAMPs or RNA) and adjuvant can be packaged and supplied in the same vial or can be packaged in separate vials and mixed before use. The compositions herein and adjuvant are typically packaged with a label indicating the intended therapeutic application. If the compositions and adjuvant are packaged separately, the packaging typically includes instructions for mixing before use. The choice of an adjuvant and/or carrier depends on the stability of the composition containing the adjuvant, the route of administration, the dosing schedule, the efficacy of the adjuvant for the species being treated, and, in humans, a pharmaceutically acceptable adjuvant is one that has been approved or is approvable for human administration by pertinent regulatory bodies. Optionally, two or more different adjuvants can be used simultaneously.

Administration of the compositions described herein comprising PAMP preparations or RNA, in pure form or in an appropriate pharmaceutical composition, can be carried out via any of the accepted modes of administration of agents for serving similar utilities. The pharmaceutical compositions can be prepared by combining the PAMP preparation or RNA (or both) containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition. Delivery of compositions comprising PAMP preparations and compositions comprising RNA is also described in further detail elsewhere herein. Administration may be achieved by a variety of different routes, including oral, parenteral, nasal, intravenous, intradermal, subcutaneous, intratumoral, intranodal or topical. Preferred modes of administration depend upon the nature of the condition to be treated or prevented. In certain embodiments, an amount that, following administration, induces an immune response is considered effective. In certain embodiments, an amount that, following administration, reduces, inhibits, prevents or delays the progression and/or metastasis of a cancer is considered effective.

In certain embodiments, the amount administered is sufficient to result in clinically relevant reduction in symptoms of a particular disease indication known to the skilled clinician.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The compositions herein may be administered one time, or may be administered multiple times at intervals of time as described elsewhere. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intratumoral, intranodal, intrasternal injection or infusion techniques. Pharmaceutical compositions according to certain embodiments of the present invention are formulated so as to allow the active ingredients contained therein to be bioavailable upon administration of the composition to a subject. Methods of preparing dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the compositions of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

Delivery of the aerosol includes the necessary container, activators, valves, subcontainers, and the like, which together may form a kit. One of ordinary skill in the art, without undue experimentation may determine preferred aerosols.

The PAMP preparations and/or RNA provided herein can be packaged as kits. Kits can optionally include one or more components such as instructions for use, devices, and additional reagents, and components, such as tubes, containers and syringes for practice of the methods. Exemplary kits can include the PAMP preparations and/or RNA provided herein, and can optionally include instructions for use, a device for detecting a PAMP and/or RNA in a subject, a device for administering the compositions comprising the adjuvant and/or RNA to a subject, and a device for administering a compound to a subject.

A kit may also contain instructions. Instructions typically include a tangible expression describing the PAMP or RNA and, optionally, other components included in the kit, and methods for administration, including methods for determining the proper state of the subject, the proper dosage amount, and the proper administration method, for administering the virus. Instructions can also include guidance for monitoring the subject over the duration of the treatment time.

Kits provided herein also can include a device for administering a PAMP or RNA preparation to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a PAMP or RNA of the kit will be compatible with the adjuvant or RNA of the kit; for example, a needle-less injection device such as a high pressure injection device can be included in kits with viruses not damaged by high pressure injection, but is typically not included in kits with a PAMP or RNA damaged by high pressure injection.

Kits provided herein also can include a device for administering a compound, such as an antigen or an immunomodulatory molecule or other active agent, to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically the device for administering the compound of the kit will be compatible with the desired method of administration of the compound.

Kits provided herein also can include a device for administering a composition comprising RNA encoding a protein of interest, such as an immunomodulatory molecule or other active agent, to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically, the device for administering the compositions comprising RNA of the kit will be compatible with the desired method of administration of the compositions.

In certain embodiments, the present disclosure provides a kit comprising (a) a first composition comprising a PAMP preparation; and (b) a second composition comprising a first RNA encoding an immunomodulatory molecule (e.g., IL12). In some embodiments of the kits, the RNA is a srRNA. In this regard, the srRNA may be derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

In certain embodiments of the kits provided herein, the second composition comprises a cationic lipid, an ionizable cationic lipid, a liposome, a lipid nanoparticle, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, or a cationic nanoemulsion. In one particular embodiment, the second composition comprises a lipid nanoparticle or a linear polyethylenimine derivative (e.g., JETPEI).

### Administration Regimens/Methods of Inducing an Immune Response

Inducing an immune response using methods and compositions described herein may be accomplished by employing a variety of different administration regimens. An exemplary, nonexhaustive list of administration regimens is presented in Table 1. These and additional embodiments of the methods for inducing an immune response are described in greater detail below and herein. Exemplary embodiments according to the present disclosure include GLA as the PAMP and a srRNA encoding IL-12 as the RNA.

TABLE 1
Exemplary Administration Regimens

| 1^{st} Admin | 2^{rd} Admin | 3^{rd} Admin |
|---|---|---|
| PAMP | RNA | None |
| PAMP | RNA | RNA |
| PAMP | RNA | PAMP |
| PAMP + RNA | PAMP + RNA | None |
| PAMP + RNA | PAMP + RNA | PAMP + RNA |
| PAMP + RNA | RNA | None |
| PAMP + RNA | RNA | RNA |
| PAMP + RNA | None | None |
| PAMP + RNA | PAMP | PAMP |
| PAMP + RNA | PAMP | RNA |
| RNA | PAMP | None |
| RNA | PAMP | RNA |
| RNA | PAMP | PAMP |

In certain embodiments of the methods for inducing an immune response, the RNA used in the second composition is a self-replicating RNA. Generally, the composition comprising RNA is formulated as described in further detail elsewhere herein.

In various embodiments and further to the exemplary regimens provided in Table 1, and described herein, certain regimens may include multiple (i.e., one, two, three, four, five, six, seven, eight, nine or ten) priming administrations (administrations of a first composition), and/or multiple (i.e., one, two, three, four, five, six, seven, eight, nine or ten) boosting administrations (administrations with a second composition), and/or multiple (i.e., one, two, three, four, five, six, seven, eight, nine or ten) secondary boosting administrations. Additionally, as described herein, any of the aforementioned compositions may include an adjuvant.

In specific embodiments, methods comprise administering the compositions (i.e., the compositions comprising an PAMP and compositions comprising RNA) more than once to the subject. In particular embodiments, a composition is administered at least two, at least three, at least four, at least five, or more times (e.g., twice (two times), three times, four times, five times, or more) to the subject. Stated another way, multiple doses (i.e., 2, 3, 4, 5, 6, 7, 8, 9, 10, or more doses) of the first and/or second composition are administered to the subject. When a first composition is administered multiple times (i.e., twice (two times), three times, four times, five times, or more), each administration of the first composition may be consecutive and, in certain embodiments, each and all administrations of the first composition are prior to administration of a second composition. In another embodiment, when a first composition is administered multiple times (i.e., twice (two times), three times, four times, five times, or more), each administration of the first composition may be consecutive and, in certain embodiments, each administration of the first composition is followed by administration of a second composition. In this regard, the first and second compositions may be administered alternatingly. In certain embodiments, a first composition is administered multiple times, such as two or three times, followed by administration of the second composition. In one embodiment, a first composition is administered multiple times, such as two or three times, followed by two consecutive administrations of the second composition. Following are non-limiting exemplary administration regimens contemplated herein. Exemplary embodiments according to the present disclosure include GLA as the PAMP and a srRNA encoding IL-12 as the RNA. (C=PAMP+RNA):
1) PAMP-RNA-PAMP-RNA
2) RNA-PAMP-RNA-PAMP
3) PAMP-RNA-RNA-RNA
4) PAMP-RNA-RNA-RNA-RNA
5) PAMP-PAMP-RNA-RNA
6) (PAMP-RNA-PAMP-RNA)×2
7) (RNA-PAMP-RNA-PAMP)×2
8) (PAMP-RNA-RNA-RNA)×2
9) (PAMP-PAMP-RNA-RNA)×2
10) PAMP-PAMP-RNA-PAMP-RNA-(PAMP Q8W up to a year)
11) C—C-RNA-RNA
12) C-RNA-RNA-RNA
13) C-RNA-RNA-RNA-(PAMP 8QW up to a year)
14) C—C-RNA-RNA-(PAMP 8QW up to a year)
15) C—C-PAMP-PAMP-RNA-RNA

As noted elsewhere, the time period between each administration of the first and second administration can vary. In one embodiment, the first immunization is comprised of concurrent administration of the first composition and the second composition (e.g., a combination of the first and the second compositions (e.g., GLA+RNA). In this regard, concurrent administration means generally at the same time (e.g., within minutes or hours or on the same day) but the two compositions may be administered at different anatomical locations and/or by different routes. Thus, concurrent does not necessarily mean that the two compositions are given in a single administration although that is contemplated. The term ‘concurrently’ refers to events that occur within 30 minutes, one hour, or even within 2, 3, 4, 5, 6, 7, 8, 9, 10, 11, 12 or 24 hours. Generally, concurrent administrations occur within 24 hours (e.g., are on the same day).

The time interval between the first administration and the second or subsequent administration(s) is discussed in greater detail herein and is selected on the basis of results from pre-clinical and/or clinical studies.

In certain embodiments of the present disclosure, the interval between the administration of one or more pairs of consecutive doses of the first composition (and/or consecutive doses of the second composition) is from about 1 to 180 days, such as about 5 to 120 days, and includes such embodiments from about 7 to 15 days or 15 to 30 days, and from about 7 to 14 days, 14 to 21 days, 21 to 28 days, 28 to 35 days, 35 to 45 days, 45 to 60 days, 60 to 75 days, 75 to 90 days, or 90 to 120 days.

With respect to the methods described herein that include sequential administration of the first composition and the second composition, or multiple consecutive administrations of either composition, the time interval between doses can be readily determined by a person skilled in the art practicing clinical trials. The dosing regimen for human subjects may also be informed by results from pre-clinical studies and knowledge in the art. In certain embodiments, the time interval between any given administration and any other administration of the same or a different composition may be at least one, two, three, four, five, six, or seven days or one, two, three, four, five, six, seven, or eight weeks, or may be at least one, two, three, four, five, six, seven, eight, nine, ten, or eleven months, or at least one, two, three, or four years. In certain embodiments, the first and second compositions may be administered to the subject a second, a third, a fourth, or fifth time. The time interval between any given administration and any other administration of the same or a different composition may be the same or different. The time intervals as described herein between administrations of the same or different compositions pertain to any of the administration regimens described herein (including, for example, the regimens provided in Table 1).

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary). In general, an appropriate dose and treatment regimen provide the proteins encoded by the sequences of interest and optionally an adjuvant or other additional active agent as detailed herein in an amount sufficient to provide therapeutic and/or prophylactic benefit. Therapeutic and/or prophylactic benefit resulting from therapeutic treatment and/or prophylactic or preventative methods include, for example an improved clinical outcome, wherein the object is to prevent or slow or retard (lessen) an undesired physiological change or disorder, or to prevent or slow or retard (lessen) the expansion or severity of such disease or disorder. Beneficial or desired clinical results from treating a subject include, but are not limited to, abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can also mean prolonging survival when compared to expected survival if a subject were not receiving treatment. Subjects in need of the methods and compositions described herein include those who already have the disease or disorder as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit. The design and execution of the appropriate preclinical studies and clinical studies can be readily performed by persons skilled in the relevant art(s).

Compositions comprising PAMP preparations and/or RNA (e.g., srRNA) may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human subject including a non-human mammalian subject.

With respect to administration of a PAMP preparation or a composition comprising an RNA (e.g., srRNA), or any of the administration regimens thereof described herein, a therapeutically effective amount provides an amount of the composition comprising the PAMP and/or RNA which is capable of producing a medically desirable result (i.e., a sufficient amount of the immunogen/antigen or other protein of interest is expressed to induce or enhance the immune response; humoral and/or cell-mediated response, including a cytotoxic T cell response) in a statistically, biologically, clinically, and/or significant manner) in a treated human or non-human animal. As is well known in the medical arts, the dosage for any one patient depends upon many factors, including the patient's size, body surface area, age, the particular compound to be administered, sex, time and route of administration, general health, and other drugs being administered concurrently. Doses will vary, but, in one embodiment, a dose for administration of a PAMP preparation is sufficient to provide approximately 10⁶ to 10¹² vector genomes comprising the sequence of interest.

The first and/or second compositions, in the context of any aspects of the present disclosure may be administered orally, parenterally, by inhalation spray, topically, rectally, nasally, buccally, vaginally or via an implanted reservoir. The term parenteral as used herein includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial and sublingual injection or infusion techniques. Also envisioned are embodiments where the first and/or second composition is administered intranodally or intra-tumorally. In particular embodiments the first composition and/or the second composition is administered by intratumoral injection. Sterile injectable forms of the inventive pharmaceutical compositions may be aqueous or oleaginous suspension. These suspensions may be formulated according to techniques known in the art using suitable dispersing or wetting agents and suspending agents.

The administration regimens of the present disclosure are used in methods for inducing an immune response, in particular for the treatment of a variety of diseases. Therefore, the present disclosure provides methods of treating a variety of diseases by administering the first and second compositions according to any of the regimens described herein.

Immune Responses

As described herein, methods are provided for inducing an immune response, in particular an immune response against cancer antigens present in tumors. In this regard, the administration of the PAMP preparations into a tumor can be referred to as a tumor immunization in that the PAMP preparation activates the immune system via one or more innate immune receptors (e.g., TLR4 or other TLR receptor) on cells within the tumor and eventually activates the adaptive immune response via T cells present in the tumor or a draining lymph node. Thus, the present disclosure provides methods for inducing an antigen-specific immune response, and in certain embodiments, the immune response induced is a strong polyfunctional T cell response, including in particular, a polyfunctional CD8 T cell response.

As described elsewhere herein, an antigen refers typically to a substance which may be recognized by the immune system and may be capable of triggering an antigen-specific immune response, e.g. by formation of antibodies or antigen-specific T-cells as part of an adaptive immune response. Other terms used in this context include immunogen. An antigen may be a peptide presented to T cells within a tumor. In certain embodiments, an antigen is a neoantigen expressed by the tumor cells.

Whether an immune response is induced and the type of immune response induced in a host or subject may be determined by any number of well-known immunological methods described herein and with which those having ordinary skill in the art will be familiar. Methods and techniques for determining the presence and level of an immune response include, for example, fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, immunoassays, (such as enzyme-linked immunosorbant assays (ELISA), radioimmunoassay, immunoblotting, immunohistochemistry, and the like), surface plasmon resonance, cell-based assays such as those that use reporter genes, and functional assays (e.g., assays that measure immune function and immunoresponsiveness such as ELISPOT or intracellular cytokine staining (ICS) assays).

Such assays include, but need not be limited to, in vivo or in vitro determination of the presence and level of soluble antibodies, soluble mediators such as cytokines (e.g., IFN-γ, IL-2, IL-4, IL-10, IL-12, IL-6, IL-23, TNF-α, and TGF-β), lymphokines, chemokines, hormones, growth factors, and the like, as well as other soluble small peptide, carbohydrate, nucleotide and/or lipid mediators. Immunoassays also include determining cellular activation state changes by analyzing altered functional or structural properties of cells of the immune system, for example, cell proliferation, altered motility, induction of specialized activities such as specific gene expression or cytolytic behavior; cell maturation, such as maturation of dendritic cells in response to a stimulus; alteration in relationship between a Th1 response and a Th2 response; cellular differentiation by cells of the immune system, including altered surface antigen expression profiles or the onset of apoptosis (programmed cell death). Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

Determining the presence and/or level of antibodies that specifically bind to an immunogen and the respective designated antigen of interest may be determined using any one of several immunoassays routinely practiced in the art, including but not limited to, ELISAs, immunoprecipitation, immunoblotting, countercurrent immunoelectrophoresis, radioimmunoassays, dot blot assays, inhibition or competition assays, and the like (see, e.g., U.S. Pat. Nos. 4,376,110 and 4,486,530; Harlow et al., Antibodies: A Laboratory Manual, Cold Spring Harbor Laboratory (1988)). Assays may also be performed to determine the class and isotype of an antibody that specifically binds to an immunogen. Antibodies (polyclonal and/or monoclonal or antigen-binding fragments thereof), which specifically bind to an immunogen and which may be used as controls in immunoassays detecting an antibody-specific immune response in an immunized subject, may generally be prepared by any of a variety of techniques known to persons having ordinary skill in the art. See, e.g., Harlow et al., Antibodies: A Laboratory Manual, Cold Spring Harbor Laboratory (1988); Peterson, ILAR J. 46:314-19 (2005); (Kohler et al., Nature, 256:495-97 (1976); Kohler et al., Eur. J. Immunol. 6:511-19 (1975); Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); Antibodies: A Laboratory Manual, Harlow and Lane (eds.), Cold Spring Harbor Laboratory Press (1988); see also, e.g., Brand et al., Planta Med. 70:986-92 (2004); Pasqualini et al., Proc. Natl. Acad. Sci. USA 101:257-59 (2004). The immunogen, or immunogenic fragments thereof, or a cell or particle bearing the immunogen or immunogenic fragment thereof may be used for immunizing an animal for production of either polyclonal antibodies or monoclonal antibodies.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as spleen cells or cells from lymph nodes, peripheral blood mononuclear cells, or from tumors, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

The level of a CTL immune response and the level of a memory CD4 T cell response may be determined by any one of numerous immunological methods described herein and routinely practiced in the art. The level of a CTL immune response may be determined prior to administration of any one of the compositions, RNA, vectors, or vector particle preparations described herein and then used for comparison with the level of CTL immune response at an appropriate time point after one or more administrations of the compositions, RNA, vectors, or vector particle preparations that provide memory CD4 T cell help. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

As used herein, a binding partner or an antibody is said to be “immunospecific,” “specific for” or to “specifically bind” an immunogen of interest if the antibody reacts at a detectable level with the immunogen or immunogenic fragment thereof, preferably with an affinity constant, Ka, of greater than or equal to about 104 M−1, or greater than or equal to about 105 M−1, greater than or equal to about 106 M−1, greater than or equal to about 107 M−1, or greater than or equal to 108 M−1. Affinity of an antibody for its cognate antigen is also commonly expressed as a dissociation constant KD, and an antibody specifically binds to the immunogen of interest if it binds with a KD of less than or equal to 10-4 M, less than or equal to about 10-5 M, less than or equal to about 10-6 M, less than or equal to 10-7 M, or less than or equal to 10-8 M.

Affinities of binding partners or antibodies can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to a binding partner (or ligand) in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

A biological sample may be obtained from the subject for determining the presence and level of an immune response to an antigen in the subject who has received any one or more of the compositions comprising PAMP preparations and/or compositions comprising RNA described herein, according to the administration regimens described herein. A “biological sample” as used herein may be a blood sample (from which serum or plasma may be prepared), leukapheresis, biopsy specimen, tumor biopsy, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any of the compositions described herein, which biological sample is useful as a control for establishing baseline (i.e., pre-immunization) data.

With respect to all immunoassays and methods described herein for determining an immune response, a person skilled in the art will also readily appreciate and understand which controls are appropriately included when practicing these methods. Concentrations of reaction components, types of buffers, temperature, and time periods sufficient to permit interaction of the reaction components can be determined and/or adjusted according to methods described herein and with which persons skilled in the art are familiar.

Combination Treatments

The compositions and methods described herein may also be administered simultaneously with, prior to, or after administration of one or more other active agents or procedures.

Thus, in certain embodiments, also contemplated is the administration of an administration regimen herein in combination with one or more other active agents (e.g. other anti-cancer agents, or other palliative or adjunctive therapy). In certain embodiments, such active agents may be accepted in the art as a standard treatment or prevention for a particular cancer as described herein. Exemplary active agents contemplated include cytokines, growth factors, steroids, NSAIDs, DMARDs, anti-inflammatories, immune checkpoint inhibitors, chemotherapeutics, radiotherapeutics, or other active and ancillary agents.

In one embodiment, a regimen is administered in combination with one or more active agents used in the treatment of cancer, including one or more chemotherapeutic agents. Examples of such active agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer active agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors.

In another embodiment, an administration regimen as described herein is administered in combination with another immunostimulatory agent. Such immunostimulatory agents include, but are not limited to, N-acetylmuramyl-L-alanine-D-isoglutamine (MDP), glucan, IL-12, GM-CSF, interferon-γ and anti-CD40 antibodies or other antibodies that bind to and activate co-stimulatory pathways (e.g., CD28, ICOS, OX40, CD27 and the like).

In one embodiment, an administration regimen as described herein is administered in combination with one or more immune checkpoint inhibitors. Immune checkpoints refer to a variety of inhibitory pathways of the immune system that are crucial for maintaining self-tolerance and for modulating the duration and amplitude of an immune responses. Tumors use certain immune-checkpoint pathways as a major mechanism of immune resistance, particularly against T cells that are specific for tumor antigens. (see., e.g., Pardoll, 2012 Nature 12:252; Chen and Mellman 2013 Immunity 39:1). The present disclosure provides immune checkpoint inhibitors that can be administered in combination with the first and/or second compositions described herein. Such combination therapies work in concert to enhance an anti-cancer immune response. Certain viruses have also developed mechanisms to co-opt immune checkpoint pathways. Therefore, in certain embodiments, such combination therapy may be used to enhance an anti-viral immune response.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55) and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), Pembrolizumab (MK-3475; PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

In a further embodiment, an administration regimen as described herein is administered in combination with other TLR4 agonists, or a TLR8 agonist, or a TLR9 agonist. Such an agonist may be selected from peptidoglycan, polyLC, CpG, 3M003, flagellin, and Leishmania homolog of eukaryotic ribosomal elongation and initiation factor 4a (LeIF).

In an additional embodiment, an administration regimen is administered in combination with a cytokine. By “cytokine” is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1 through IL-36, including, but not limited to, IL-1, IL-1alpha, IL-2, IL-3, IL-4, IL-5, IL-6, IL-7, IL-8, IL-9, IL-10, IL-11, IL-12; IL-15, IL-18, IL-21, IL-23, IL-27, TNF; and other polypeptide factors including LIF and kit ligand (KL). As used herein, the term cytokine includes proteins from natural sources or from recombinant cell culture, and biologically active equivalents of the native sequence cytokines.

In certain embodiments, the compositions disclosed herein may be administered in conjunction with a histone deacetylase (HDAC) inhibitor. HDAC inhibitors include hydroxamates, cyclic peptides, aliphatic acids and benzamides. Illustrative HDAC inhibitors contemplated for use herein include, but are not limited to, Suberoylanilide hydroxamic acid (SAHA/Vorinostat/Zolinza), Trichostatin A (TSA), PXD-101, Depsipeptide (FK228/romidepsin/ISTODAX®), panobinostat (LBH589), MS-275, Mocetinostat (MGCD0103), ACY-738, TMP195, Tucidinostat, valproic acid, sodium phenylbutyrate, 5-aza-2′-deoxycytidine (decitabine). See e.g., Kim and Bae, Am J Transl Res 2011; 3(2):166-179; Odunsi et al., Cancer Immunol Res. 2014 Jan. 1; 2(1): 37-49. Other HDAC inhibitors include Vorinostat (SAHA, MK0683), Entinostat (MS-275), Panobinostat (LBH589), Trichostatin A (TSA), Mocetinostat (MGCD0103), ACY-738, Tucidinostat (Chidamide), TMP195, Citarinostat (ACY-241), Belinostat (PXD101), Romidepsin (FK228, Depsipeptide), MC1568, Tubastatin A HCl, Givinostat (ITF2357), Dacinostat (LAQ824), CUDC-101, Quisinostat (JNJ-26481585) 2HCl, Pracinostat (SB939), PCI-34051, Droxinostat, Abexinostat (PCI-24781), RGFP966, AR-42, Ricolinostat (ACY-1215), Valproic acid sodium salt (Sodium valproate), Tacedinaline (CI994), CUDC-907, Sodium butyrate, Curcumin, M344, Tubacin, RG2833 (RGFP109), Resminostat, Divalproex Sodium, Scriptaid, and Tubastatin A (see e.g., selleckchem(dot)com).

In certain embodiments, an administration regimen is administered in combination with chloroquine, a lysosomotropic agent that prevents endosomal acidification and which inhibits autophagy induced by tumor cells to survive accelerated cell growth and nutrient deprivation. More generally, the compositions comprising heterozygous viral vectors as described herein may be administered in combination with active agents that act as autophagy inhibitors, radiosensitizers or chemosensitizers, such as chloroquine, misonidazole, metronidazole, and hypoxic cytotoxins, such as tirapazamine. In this regard, such combinations of a heterozygous viral vector with chloroquine or other radio or chemo sensitizer, or autophagy inhibitor, can be used in further combination with other cancer active agents or with radiation therapy.

In another embodiment, a regimen is administered in combination with small molecule drugs which are known to result in killing of tumor cells with concomitant activation of immune responses, termed “immunogenic cell death”, such as cyclophosphamide, doxorubicin, oxaliplatin and mitoxantrone. Furthermore, combinations with drugs known to enhance the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, furthermore proteasome inhibitors (e.g. bortezomib), shikonin (the major constituent of the root of Lithospermum erythrorhizon) and oncolytic viruses, such as TVec (talimogene laherparepvec). In other embodiments, the compositions as described herein may be administered in combination with epigenetic therapies, such as DNA methyltransferase inhbitors (e.g. Decitabine, 5-aza-2′-deoxycytidine) which may be administered locally or systemically.

In another embodiment, an administration regimen is administered in combination with one or more antibodies that increase ADCC uptake of tumor by DCs. Thus, the present invention contemplates combining compositions comprising a retroviral vector preparation with any molecule that induces or enhances the ingestion of a tumor cell or its fragments by an antigen presenting cell and subsequent presentation of tumor antigens to the immune system. These molecules include agents that induce receptor binding (such as Fc or mannose receptors) and transport into the antigen presenting cell such as antibodies, antibody-like molecules, multi-specific multivalent molecules and polymers. Such molecules may either be administered intratumorally with the composition comprising heterozygous viral vector, or administered by a different route. For example, a composition comprising heterozygous viral vector as described herein may be administered intratumorally in conjunction with intratumoral injection of rituximab, cetuximab, trastuzumab, Campath, panitumumab, ofatumumab, brentuximab, pertuzumab, Ado-trastuzumab emtansine, Obinutuzumab, anti-HER1, -HER2, or -HER3 antibodies (e.g., MEHD7945A; MM-111; MM-151; MM-121; AMG888), anti-EGFR antibodies (e.g. Nimotuzumab, ABT-806), or other like antibodies. Any multivalent scaffold that is capable of engaging Fc receptors and other receptors that can induce internalization may be used in the combination therapies described herein—e.g. peptides and/or proteins capable of binding targets that are linked to Fc fragments or polymers capable of engaging receptors.

In certain embodiments, a regimen may be further combined with an antibody that promotes a co-stimulatory signal (e.g., by blocking inhibitory pathways), such as anti-CTLA-4, or that activates co-stimulatory pathways such as an anti-CD40, anti-CD28, anti-ICOS, anti-OX40, anti-CD27 antibodies and the like.

The regimens herein may be administered alone or in combination with other known cancer treatments, such as radiation therapy, immune checkpoint inhibitors, chemotherapy or other cancer active agents, transplantation, immunotherapy, hormone therapy, photodynamic therapy, etc. The compositions may also be administered in combination with antibiotics.

As used herein and in the appended claims, the singular forms “a,” “and,” and “the” include plural referents unless the context clearly dictates otherwise. Thus, for example, reference to “an antigen” includes a plurality of such antigens, and reference to “a cell” or “the cell” includes reference to one or more cells and equivalents thereof (e.g., plurality of cells) known to those skilled in the art, and so forth. Similarly, reference to “a compound” or “a composition” includes a plurality of such compounds or compositions, and refers to one or more compounds or compositions, respectively, unless the context clearly dictates otherwise. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The following examples are offered by way of illustration, and not by way of limitation.

### EXAMPLES

### Example 1

### In Vitro Production of Potent Self-Replicating RNA

This Example outlines the process in which RNA was generated using a standard transcription protocol known in the art.

The RNA used in this example is a self-replicating RNA (srRNA) derived from an alphavirus. The alphaviral sequences (5′UTR, nonstructural proteins, 26S promoter, and 3′UTR sequences) were derived from GenBank accession number L01443.1 (version 1, Gi 323714, Apr. 23, 2010, 2:26 PM), Venezuelan equine encephalitis (VEE) virus strain TC-83 (attenuated). An illustrative wild type alphavirus sequence is also provided in SEQ ID NO:1.

srRNA is very different from typical mRNA for at least the following reasons: 1) mRNA encodes a single protein—srRNA encodes multiple viral proteins and protein of interest; 2) srRNA is usually very large ˜9-11 kb while mRNAs are typically ˜2 kb; 3) srRNA has different requirements for production and delivery. However, the srRNA functions directly as an mRNA in that it is 5′-capped and 3′ polyadenylated. Replication of the alphavirus self-replicating RNA yields high levels of a shorter, sub-genomic RNA species derived from the 3′ end of the RNA and driven from the 26S RNA dependent RNA polymerase promoter. The sub genomic RNA nucleotide sequence is translated at extremely high levels (up to 20% of total cell protein).

DNA containing the VEE replicon with the sequence of interest (e.g., GFP, IL12) inserted in place of VEE structural protein genes (see e.g., FIG. 1B SOI downstream of either NSP4 or REP), was synthesized commercially (GenScript, Piscataway, N.J.). Standard molecular biology cloning methods could also be used.

A typical protocol for making RNA from plasmid DNA used a linearized DNA template encoding for all essential RNA components, including a 5′ cap and untranslated region (UTR) and 3′ UTR and polyA tail, in addition to sequences of interest, e.g., antigens, reporter molecules, immunomodulatory proteins and/or any other sequence of interest. The DNA template was added to standard in vitro transcription reaction mix including a buffer, a ribonucleotide cocktail (adenosine triphosphate [ATP], guanosine triphosphate [GTP], cytidine triphosphate [CTP] and uridine triphosphate [UTP]) and an RNA polymerase enzyme mix. This reaction mix is incubated at an appropriate temperature to allow transcription and the RNA product was precipitated and resuspended. Additional modifications were made to improve stability and translation efficiency, including substitution of the original 5′ cap with a 5′ methylated GTP.

After RNA generation and capping, purity and integrity of the RNA product was determined by measuring ultraviolent absorbance at 260 and 280 nm, followed by analysis of a 0.8% agarose-ethidium bromide gel. High-integrity RNA appeared on agarose gels as a single distinct band at the expected size, indicating minimal degradation.

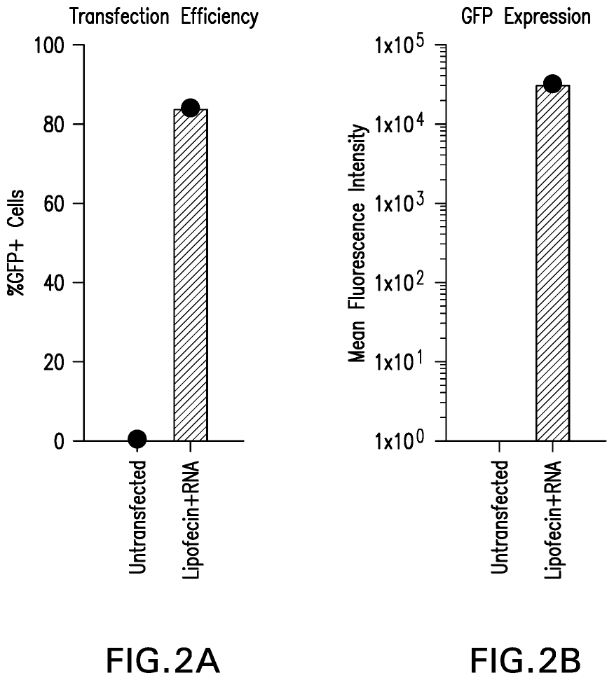

RNA potency was also assessed in BHK-21 cells (ATCC BHK-21 [C-13]). RNA was formulated with off-the-shelf transfection reagents, such as Lipofectin Transfection Reagent (ThermoFisher, Waltham, Mass.) and used to transfect adherent BHK-21 cells. Approximately 16-24 hours following transfection, cells were analyzed for expression of the gene of interest (e.g. GFP). Transfection was very efficient and resulted in high expression of the gene of interest (e.g. GFP) (FIG. 2).

### Example 2

### RNA Formulated with Polyethylenimine Forms Transfection Complexes

This Example demonstrates that RNA can be formulated with catonic polymers, such as polyethylenimine, including jetPEI (Polyplus, Illkirch, France).

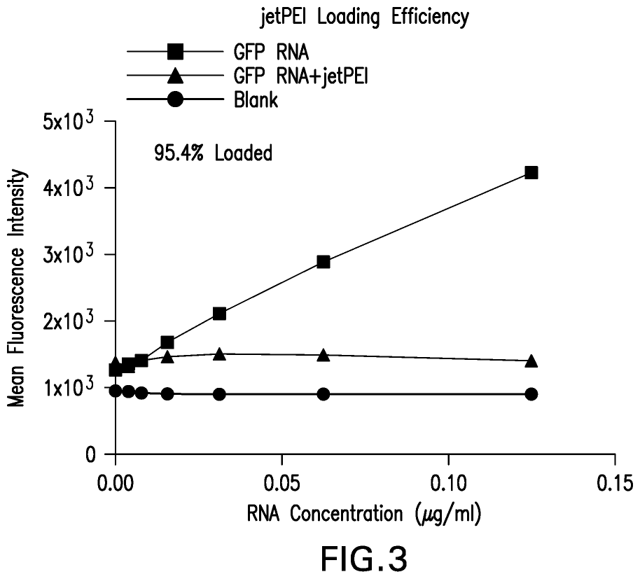

RNA was formulated with jetPEI according to the manufacturer's protocol (Polyplus). In a 5% glucose solution, RNA was mixed with jetPEI at various nitrogen to phosphate ratios, including the nitrogen to phosphate ratio of 8, which was recommended by the manufacturer. Using a Quant-iT RiboGreen RNA assay kit (ThermoFisher, Waltham, Mass.), the amount of RNA that was successfully complexed with the cationic polymer could be quantified. Cationic polymers such as jetPEI block the fluorescent nucleic acid stain from binding to the complexed RNA, allowing quantification of complexation efficiency. At a nitrogen to phosphate ratio of 8, 95% of RNA was successfully complexed with jetPEI (FIG. 3).

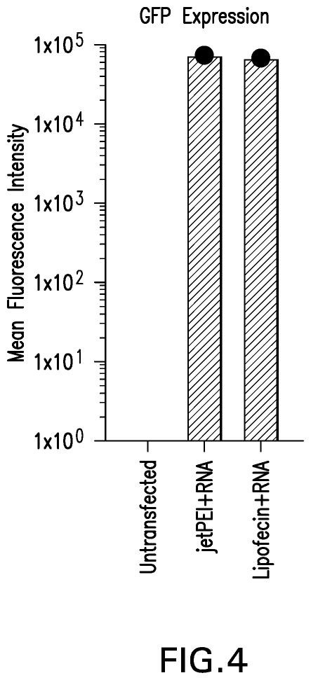

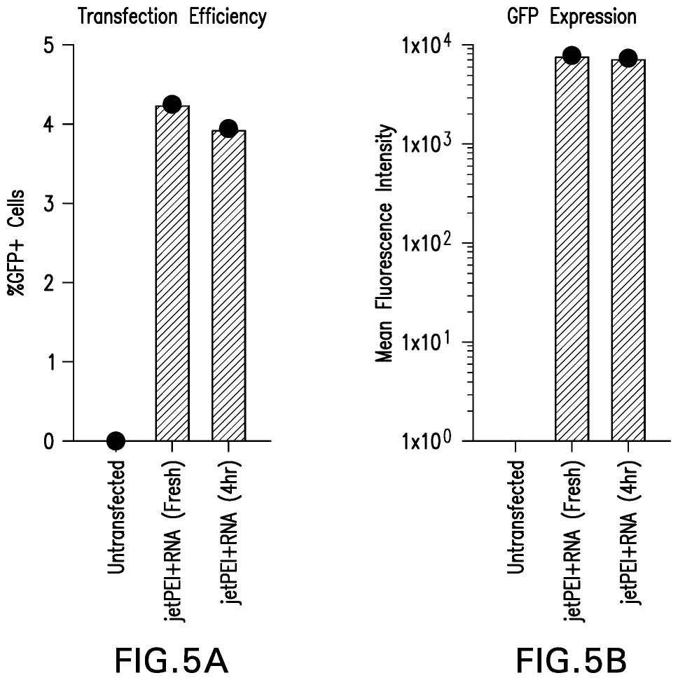

RNA formulated with jetPEI was used as a transfection agent in BHK-21 cells, and was also used to determine stability of jetPEI-RNA complexes. BHK-21 cells that were transfected with GFP-expressing RNA formulated with jetPEI expressed high levels of GFP compared to untransfected cells, similar to cells transfected using Lipofectin Transfection Reagent (FIG. 4). jetPEI transfection complexes were stable for 4 hours, indicated by efficient transfection of BHK cells (FIG. 5).

### Example 3

### In Vivo Expression and Function of Srrna/Mil12 in Mice

This example describes experiments to determine the systemic levels of IL12 expressed from srRNA and to confirm that the encoded mIL12 functioned in vivo.

IL-12 is composed of 2 disulfide linked to subunits, p35 and p40. Two different constructs were prepared with both subunits connected via an elastin linker but in different orientations: p35-elastin-p40 and p40-elastin-p35 (p35-L-p40; p40-L-p35). Transfection experiments showed that the p40-L-p35 srRNA produced higher quantities of RNA. A functional bioassay showed that both IL-12 candidates produced functional IL-12. However, the p40-L-p35 candidate was selected for continued studies.

TABLE 2
Table 2 below summarizes the experiment. All RNA was formulated in jetPEI.
In vivo srRNA-IL12 study

| | Group (n = 10) 2 | | srRNA dose | |
|---|---|---|---|---|
| | groups of 5 | srRNA | (ug/mouse) | Immunize//bleed//sac |
| 1 | Control | None | 0 | 0//3, 7//14, 21 |
| 2 | GFP-srRNA | VEE-GFP-Cap1 | 3 | 0//3, 7//14, 21 |
| 3 | IL12-srRNA 1 | VEE-IL12-Cap1 | 1 | 0//3, 7//14, 21 |
| 4 | IL12-srRNA 3 | VEE-IL12-Cap1 | 3 | 0//3, 7//14, 21 |
| 5 | IL12-srRNA weekly | VEE-IL12-Cap1 | 1/injection | 0, 7, 14//3, 7//14, 21 |

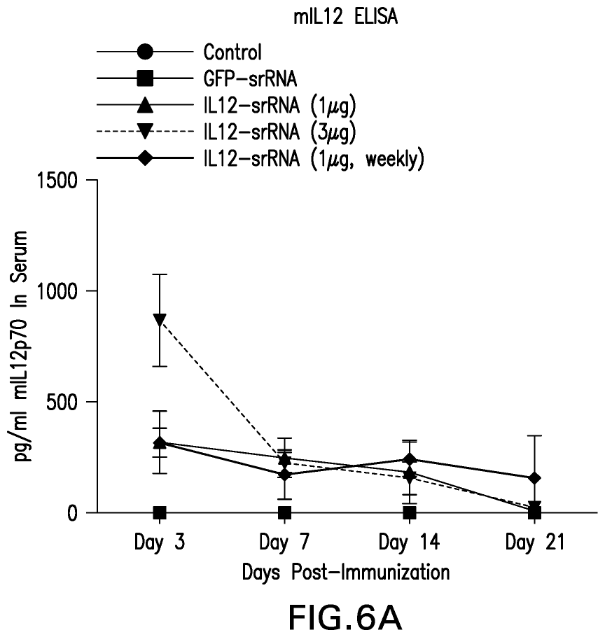

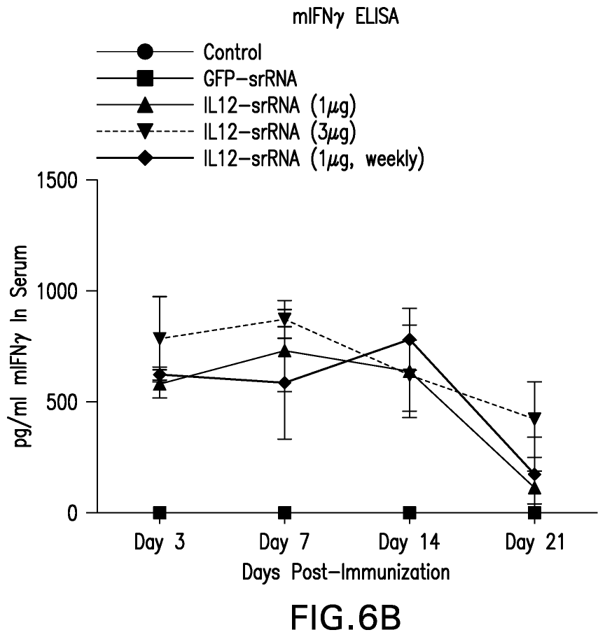

As shown in FIG. 6A, IL12 can be detected at high levels in the serum of mice immunized with 1 ug or 3 ug of IL12/srRNA formulated in jetPEI. As shown in FIG. 6B, the IL12 expressed by the jetPEI-srRNA is active and induces IFNγ production that can be detected in mouse serum.

### Example 4

### In Vivo Anti-Cancer Effect of G100±RNA/Mil12 in Murine Tumor Models

This Example demonstrates the therapeutic efficacy of G100±RNA/murine (m) IL12 in the B16 melanoma, the CT26 colon carcinoma model and the 4T1 mammary carcinoma models.

In the B16 model, on Day 0, C57BL/6 mice (n=8-10 per group) were inoculated with 1.5×10⁵ B16F10 cells, subcutaneously in the right flank. When tumors measured >4 mm² (Day 7), mice were treated with intratumoral G100 (10 μg GLA in 2% SE), intratumoral naked RNA expressing murine IL12 (nRNA/mIL12, 8 ug), intratumoral RNA/mIL12 formulated with jetPEI (fRNA/mIL12), G100+nRNA/mIL12, or G100+fRNA/mIL12. G100 administration was continued every 3-4 days thereafter, whereas RNA/mIL12 was administered once per week. Intratumoral treatments were stopped once tumors completely regressed.

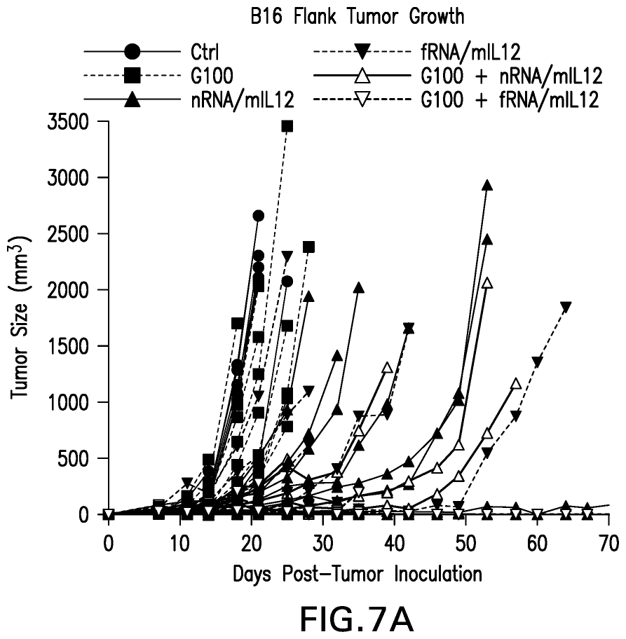

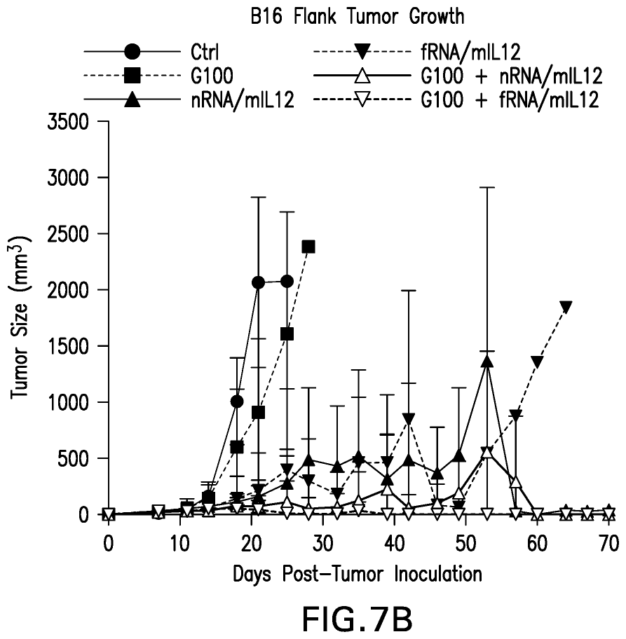

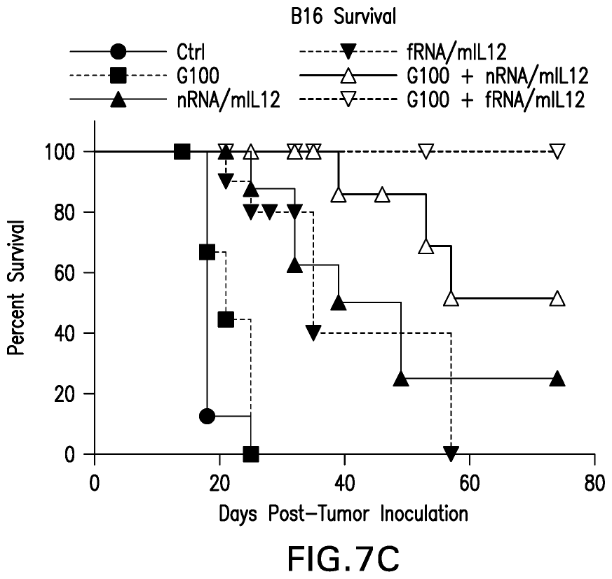

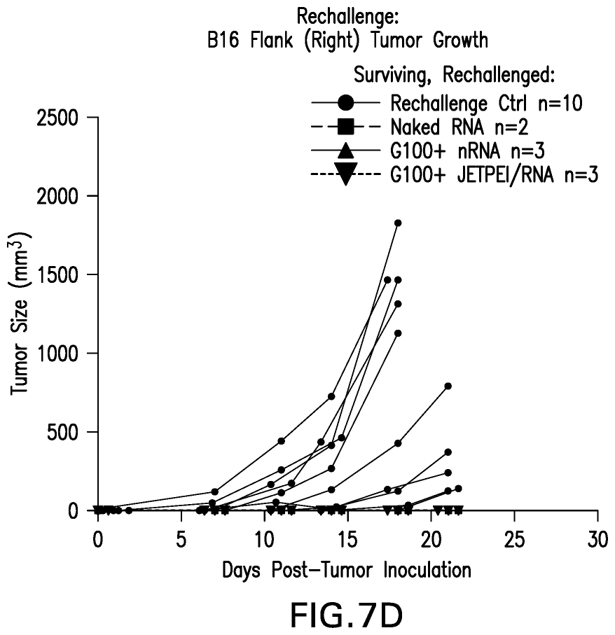

By Day 21 post-tumor implantation, G100, RNA/mIL12 (naked or formulated), or G100+RNA/mIL12 (naked or formulated), all delayed B16 tumor growth significantly (p<0.0001, ANOVA; FIG. 7A and FIG. 7B). No tumor regression was observed in mice treated with G100 alone, whereas tumor regression was observed in 1/9 mice treated with nRNA/mIL12, 2/10 mice with fRNA/mIL12, 5/10 mice with G100+nRNA/mIL12, and 7/10 mice with G100+fRNA/mIL12, all leading to significant improvement in survival (p<0.0001, Mantel-Cox) (FIG. 7C). On Day 74, the original B16 study continued with initiation of a rechallenge study. The surviving mice in groups treated with G100+nRNA/mIL12 and G100+fRNA/mIL12 (3 mice each group) and 2 surviving mice in the group treated with nRNA/mIL12 were given single-sided B16F10 tumors (2E6 cells), and 10 rechallenge control (naïve) mice were given the same. As shown in FIG. 7D, all rechallenged mice are still surviving at day 21 (experiment is ongoing) surprisingly indicating that the treatments induced the generation of immunological memory.

In the CT26 model, on Day 0, BALB/c mice (n=9-10 per group) were inoculated with 1.5×10⁵ and 0.5×10⁵ CT26 cells, subcutaneously in the right and left flank, respectively. When the right flank tumors measured >4 mm² (Day 7), only the right flank tumors were treated with intratumoral G100 (10 μg GLA in 2% SE), intratumoral nRNA/mIL12 (8 μg), intratumoral fRNA/mIL12, G100+nRNA/mIL12, or G100+fRNA/mIL12. G100 administration was continued every 3-4 days thereafter, whereas RNA/mIL12 was administered once per week. Intratumoral treatments were stopped once tumors completely regressed.

By Day 25 post-tumor implantation, G100 alone did not significantly impact growth of the treated (right) tumor, whereas RNA/mIL12 (naked or formulated) or G100+RNA/mIL12 (naked or formulated), all significantly (p=0.0028, p=0.0001, p<0.0001, p<0.0001, respectively; ANOVA) delayed its growth (FIG. 8A and FIG. 8B). Tumor regression was observed in 4/9 mice treated with nRNA/mIL12, 3/10 mice with fRNA/mIL12, 5/9 mice with G100+nRNA/mIL12, and 5/10 mice with G100+fRNA/mIL12. G100 alone did not significantly improve survival, whereas RNA/mIL12 (naked or formulated) or G100+RNA/mIL12 (naked or formulated), all significantly did (p=0.0049, p=0.0037, p=0.0001, p=0.0229, respectively; Mantel-Cox) (FIG. 8C). Unfortunately, none of the tested regimens significantly impacted growth of the untreated (left) tumors (FIGS. 8A and 8B), which was reflected in the overall survival curve (FIG. 8C). Mice with completely regressed treated tumors that would have otherwise enjoyed long-term tumor-free survival were sacrificed due to the uncontrolled growth of the untreated tumors.

Given the instability of naked (i.e., unformulated) RNA molecules, it was surprising that naked RNA/mIL12 mediated similar therapeutic efficacy as formulated RNA/mIL12 in both B16 and CT26 models. This may potentially be due to a combination of factors, including but not limited to 1) the dose of RNA; 2) the type of formulation used.

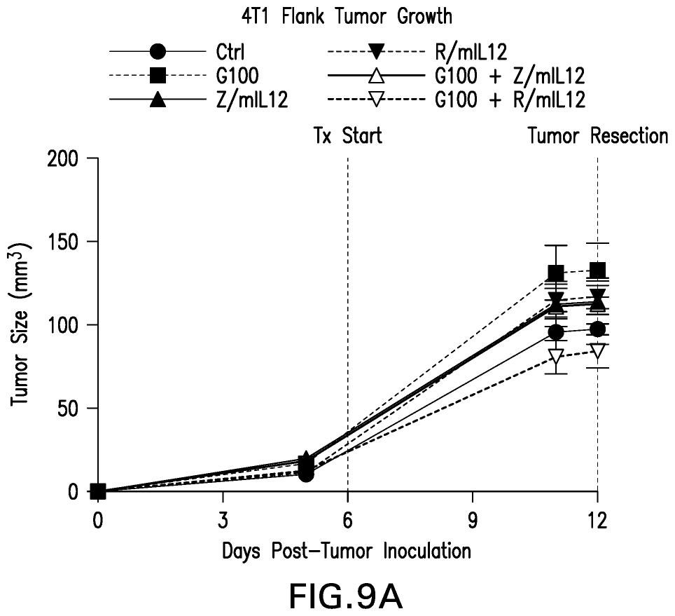

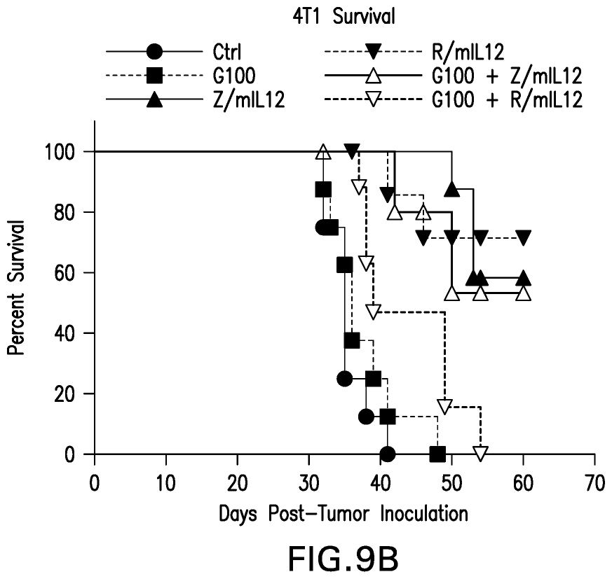

In the 4T1 model, on Day 0 BALB/c mice (n=8 per group) were inoculated with 1.0×10⁵ 4T1 cells, subcutaneously in the right flank. When tumors measured >4 mm² (Day 6), mice were treated with intratumoral G100 (20 μg GLA in 2% SE), intratumoral ZVex expressing mIL12 (Z/mIL12) (DC-targeting lentiviral vector as described for example in WO2017083291), intramuscular RNA/mIL12 formulated with jetPEI (R/mIL12), G100+Z/mIL12, or G100+R/mIL12. G100 was administered again on Day 11, followed by 4T1 tumor resection on Day 12 (FIG. 9A). G100 administration was continued thrice per week thereafter, intramuscularly, whereas RNA/mIL12 was administered once per week, until the end of the study. Due to the removal of tumors, study endpoint was assessed only by survival, which was monitored daily. Death was recorded if a mouse passed or was sacrificed due to weight loss of greater than 20% of its original body weight.

By Day 60 post-tumor implantation (Day 48 post-tumor resection), mice treated with either Z/mIL12 or R/mIL12 had the best improvement in survival (p<0.0001 and p=0.0001, respectively) (FIG. 9B). G100 alone did not significantly impact survival, and when combined with either Z/mIL12 or R/mIL12, it also did not significantly improve upon Z/mIL12- or R/mIL12-mediated efficacy. Given that upon the removal of tumors, G100 could no longer be administered intratumorally, the lack of synergy observed between G100 and Z/mIL12 or R/mIL12 was likely due to the absence of an inflammatory tumor microenvironment and continual release of tumor antigens, mediated by intratumoral G100. We thus hypothesize that intratumoral G100 is required for exposing antigen-presenting cells to tumor antigens, and intratumoral Z/mIL12 or R/mIL12 likely promote efficient activation of immune cells against the tumor antigens.

The above data demonstrate that G100 and RNA/mIL12 combine synergistically to control tumor growth. Surprisingly, the B16 model demonstrated that the combination of G100+nRNA/IL12 or fRNA/IL12 resulted in the generation of immunological memory. In this experiment, naked RNA/IL12 also appears to have induced immunological memory. This experiment is ongoing and data from later timepoints may provide additional insight into the strength of the immune response following tumor rechallenge.

In embodiment 1, a method for inhibiting tumor growth in a subject is provided, the method includes (a) administering to the subject at least one dose of a first composition comprising a pathogen-associated molecular pattern (PAMP) molecule and (b) administering to the subject at least one dose of a second composition comprising a first RNA encoding at least one immunostimulatory molecule, thereby inducing an immune response.

In embodiment 2, a method for treating cancer in a subject is provided, the method including (a) administering to the subject at least one dose of a first composition comprising a PAMP molecule; and (b) administering to the subject at least one dose of a second composition comprising a first RNA encoding at least one immunostimulatory molecule, thereby inducing an immune response.

In embodiment 3, a method for inducing an immune response in a subject, the method including (a) administering to the subject at least one dose of a first composition comprising a PAMP molecule; and (b) administering to the subject at least one dose of a second composition comprising a first RNA encoding at least one immunostimulatory molecule, thereby inducing an immune response.

In embodiment 4, the immunostimulatory molecule of embodiment 1-3 is IL-12.

In embodiment 5, the IL-12 of embodiment 4 is a single chain IL-12 (scIL-12).

In embodiment 6, the method of embodiment 5 wherein the scIL-12 comprises p35-L-p40.

In embodiment 7, the method of embodiment 5 wherein the scIL-12 comprises p40-L-p³⁵.

In embodiment 8, the method of any of embodiments 1-7 wherein the PAMP molecule is an innate immune receptor agonist.

In embodiment 9, the method of embodiment 8 wherein the innate immune receptor agonist is a glycan or a glycoconjugate.

In embodiment 10, the method of any of embodiments 1-9 wherein the PAMP molecule is a lipoprotein or lipopeptide.

In embodiment 11, the method of any of embodiments 1-10 wherein the PAMP molecule is a TLR agonist.

In embodiment 12, the method of any of embodiments 1-11 wherein the PAMP molecule is a TLR4 agonist.

In embodiment 13, the method of any of embodiments 1-12 wherein the PAMP molecule is a TLR1, TLR2, TLR3, TLR4, TLR5, TLR6, TLR7, TLR8, TLR9, TLR10, TLR11, TLR12 or TLR13 agonist.

In embodiment 14, the method of any of embodiments 1-13 wherein the PAMP is a TLR4 agonist and is an aqueous or oil in water emulsion formulation of a monophosphoryl lipid A.

In embodiment 15, the method of any of embodiments 1-14 wherein the PAMP is a TLR4 agonist and is an aqueous or oil in water emulsion formulation of glucopyranosyl lipid A (GLA).

In embodiment 16, the method of any of embodiments 1-15 wherein the first composition and the second composition are administered by a route independently selected from the group consisting of intratumorally, intradermally, intravenously, subcutaneously, intranodally and intramuscularly.

In embodiment 17, the method of claim 16 wherein the first and second compositions are administered intratumorally.

In embodiment 18, the method of any of embodiments 1-17 wherein the first composition and the second composition are administered concurrently.

In embodiment 19, the method of any of embodiments 1-18 wherein the first composition and the second composition are administered concurrently and at least two doses of each composition are administered.

In embodiment 20, the method of any of embodiments 1-19 wherein the first composition and the second composition are administered sequentially.

In embodiment 21, the method of any of embodiments 1-20 wherein the first composition and the second composition are administered sequentially and at least two doses of each composition are administered.

In embodiment 22, the method of any of embodiments 1-21 wherein the second composition is administered prior to administration of the first composition.

In embodiment 23, the method of any of embodiments 1-22 wherein the first composition and the second composition are administered by the same route.

In embodiment 24, he method of any of of any of embodiments 1-23 wherein the first composition and the second composition are administered by different routes.

In embodiment 25, tThe method of any of any of embodiments 1-24 wherein the first composition and the second composition are administered sequentially and by the same route.

In embodiment 26, the method of any of embodiments 1-25 wherein the first composition and the second composition are administered sequentially and by different routes.

In embodiment 27, the method of any of embodiments 1-26 wherein at least two doses of the first composition are administered or wherein at least two doses of the second composition are administered.

In embodiment 28, the method of any of embodiments 1-27 wherein (a) two doses; (b) three doses; (c) four doses; or (d) five doses of the first composition are administered.

In embodiment 29, the method of any of embodiments 1-28 wherein two doses of the first composition are administered prior to administration of the second composition.

In embodiment 30, the method of any of embodiments 1-29 wherein the RNA is a self-replicating RNA (srRNA).

In embodiment 31, the method of embodiment 30 wherein the srRNA is derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

In embodiment 32, the method of any of embodiments 1-31 wherein the second composition comprises a cationic lipid, an ionizable lipid, a liposome, a nanoparticle, a lipid nanoparticle, a cochleate, a virosome, an immune-stimulating complex, a microparticle, a microsphere, a unilamellar vesicle, a multilamellar vesicle, an oil-in water emulsion, a water-in-oil emulsion, an emulsome, a polycationic peptide, or a cationic nanoemulsion.

In embodiment 33, the method of embodiment 32 wherein the second composition comprises a lipid nanoparticle.

In embodiment 34, the method of any of embodiments 1-33 wherein the second composition comprises a polyethylenimine derivative.

In embodiment 35, the method of embodiment 34 wherein the polyethylenimine derivative comprises JETPEI.

In embodiment 36, the method of any of embodiments 1-37 wherein the second composition further comprises an adjuvant.

In embodiment 37, the method of any of embodiments 1-36 wherein the second composition comprises a second, third or fourth RNA encoding an additional immunomodulatory molecule.

In embodiment 38, the method of any of embodiments 1-37 further comprising administering one or more additional active agents or treatments.

In embodiment 39, the method of embodiment 38 wherein the one or more additional active agents or treatments is selected from the group consisting of an immune checkpoint inhibitor, an antibody that activates a co-stimulatory pathway, a cancer chemotherapy, and radiation therapy.

In embodiment 40, a kit is provided including (a) a first composition comprising PAMP molecule; and (b) a second composition comprising a first RNA encoding at least one immunostimulatory molecule.

In embodiment 41, product is provided that includes (a) a first composition comprising a PAMP molecule; and (b) a second composition comprising a first RNA encoding at least one immunostimulatory molecule, for use in a method selected from the group consisting of (i) inhibiting tumor growth in a subject, (ii) treating cancer in a subject, and (iii) inducing an immune response in a subject.

In embodiment 42, the product of embodiment 41 wherein the immunostimulatory molecule is IL-12.

In embodiment 43, the product of embodiment 42 wherein the IL-12 is a single chain IL-12 (scIL-12).

In embodiment 44, the product of embodiment 43 wherein the scIL-12 comprises p35-L-p40.

In embodiment 45, the product of embodiment 43 wherein the scIL-12 comprises p40-L-p35.

In embodiment 46, the product of any of embodiments 41-45 wherein the PAMP molecule is an innate immune receptor agonist.

In embodiment 47, the product of embodiment 46 wherein the innate immune receptor agonist is a glycan or a glycoconjugate.

In embodiment 48, the product of any of embodiments 41-47 wherein the PAMP molecule is a lipoprotein or lipopeptide.

In embodiment 49, the product of any of embodiments 41-47 wherein the PAMP molecule is a TLR agonist.

In embodiment 50, the product of any of embodiments 41-47 wherein the PAMP molecule is a TLR4 agonist.

In embodiment 51, the product of any of embodiments 41-47 wherein the PAMP molecule is a TLR1, TLR2, TLR3, TLR4, TLR5, TLR6, TLR7, TLR8, TLR9, TLR10, TLR11, TLR12 or TLR13 agonist.

In embodiment 52, the product of any of embodiments 41-47 wherein the a PAMP molecule is an aqueous or oil in water emulsion formulation of glucopyranosyl lipid A (GLA).

In embodiment 53, the product of any of embodiments 41-52 wherein the first composition and the second composition are administered by a route independently selected from the group consisting of intratumorally, intradermally, intravenously, subcutaneously, intranodally and intramuscularly.

In embodiment 54, the product of embodiment 53 wherein the first and second compositions are administered intratumorally.

In embodiment 55, the product of any of embodiment 41-54 wherein the first composition and the second composition are administered concurrently.

In embodiment 56, the product of any of embodiment 41-54 wherein the first composition and the second composition are administered concurrently and at least two doses of each composition are administered.

In embodiment 57, the product of any of embodiment 41-54 wherein the first composition and the second composition are administered sequentially.

In embodiment 58, the product of any of embodiment 41-54 wherein the first composition and the second composition are administered sequentially and at least two doses of each composition are administered.

In embodiment 59, the product of any of embodiment 41-54 wherein the second composition is administered prior to administration of the first composition.

In embodiment 60, the product of any of embodiment 41-59 the first composition and the second composition are administered by the same route.

In embodiment 61, the product of any of embodiment 41-59 wherein the first composition and the second composition are administered by different routes.

In embodiment 62, the product of any of embodiment 41-59 wherein the first composition and the second composition are administered sequentially and by the same route.

In embodiment 63, the product of any of embodiment 41-59 wherein the first composition and the second composition are administered sequentially and by different routes.

In embodiment 64, the product of any of embodiment 41-54 wherein at least two doses of the first composition are administered or wherein at least two doses of the second composition are administered.

In embodiment 65, the product of any of embodiment 41-54 wherein (a) two doses; (b) three doses; (c) four doses; or (d) five doses of the first composition are administered.

In embodiment 66, the product of any of embodiment 41-54 wherein two doses of the first composition are administered prior to administration of the second composition.

In embodiment 67, the product of any of embodiment 41-66 wherein the RNA is a self-replicating RNA (srRNA).

In embodiment 68, the product of embodiment 67 wherein the srRNA is derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

In embodiment 69, the product of any of embodiments 41-68 is provided wherein the second composition includes a cationic lipid, an ionizable lipid, a liposome, a nanoparticle, a lipid nanoparticle, a cochleate, a virosome, an immune-stimulating complex, a microparticle, a microsphere, a unilamellar vesicle, a multilamellar vesicle, an oil-in water emulsion, a water-in-oil emulsion, an emulsome, a polycationic peptide, or a cationic nanoemulsion.

In embodiment 70, the product of embodiment 69 is provided wherein the second composition includes a lipid nanoparticle.

In embodiment 71, the product of any of embodimetns 41-68 wherein the second composition includes a polyethylenimine derivative.

In embodiment 72, the product of embodiment 71 is provided wherein the polyethylenimine derivative includes JETPEI.

In embodiment 73, the product of any of embodiments 41-72 is provided wherein the second composition further includes an adjuvant.

In embodiment 74, the product of any of embodiments 41-73 is provided wherein the second composition comprises a second, third or fourth RNA encoding an additional immunomodulatory molecule.

In embodiment 75, the product of any of embodiments 41-74 is provided further comprising one or more additional active agents or treatments.

In embodiment 76, the product of embodiment 75 is provided wherein the one or more additional active agents or treatments is selected from the group consisting of an immune checkpoint inhibitor, an antibody that activates a co-stimulatory pathway, a cancer chemotherapy, and radiation therapy.

In embodiment 77, a method is provided for inhibiting tumor growth in a subject, the method including (a) administering intratumorally to the subject at least one dose of a first composition including GLA; and (b) administering intratumorally to the subject at least one dose of a second composition including a first RNA encoding IL-12, thereby inducing an immune response.

In embodiment 78, a method is provided for treating cancer in a subject, the method including (a) administering intratumorally to the subject at least one dose of a first composition including GLA; and (b) administering intratumorally to the subject at least one dose of a second composition including a first RNA encoding IL-12, thereby inducing an immune response.

In embodiment 79, a method is provided for inducing an immune response in a subject, the method including (a) administering intratumorally to the subject at least one dose of a first composition including GLA; and (b) administering intratumorally to the subject at least one dose of a second composition comprising a first RNA encoding IL-12, thereby inducing an immune response.

In embodiment 80, the immune checkpoint inhibitors of embodiment 39 is selected from the group consisting of: remelimumab, anti-OX40, PD-L1 monoclonal antibody, pembrolizumab, nivolumab, CT-011, BY55 monoclonal antibody, AMP224, BMS-936559, MPLDL3280A, MSB0010718C, and Yervoy/ipilimumab.

In embodiment 81, the immune checkpoint inhibitor of embodiment 39 is pembrolizumab.

In embodiment 82, the immune checkpoint inhibitor of embodiment 39 is nivolumab.

In embodiment 83, the immune checkpoint inhibitors of embodiment 76 is selected from the group consisting of: remelimumab, anti-OX40, PD-L1 monoclonal antibody, pembrolizumab, nivolumab, CT-011, BY55 monoclonal antibody, AMP224, BMS-936559, MPLDL3280A, MSB0010718C, and Yervoy/ipilimumab.

In embodiment 84, the immune checkpoint inhibitor of embodiment 76 is pembrolizumab.

In embodiment 85, the immune checkpoint inhibitor of embodiment 76 is nivolumab.

In embodiment 86, a composition including a pathogen-associated molecular pattern (PAMP) molecule and an adjuvant is provided.

In embodiment 87, the adjuvant of embodiment 86 is selected from: glucopyranosyl lipid A, an aluminum salt, 3 De-O-acylated monophosphoryl lipid A, and a lipid nanoparticle.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-2. (canceled)

3. A method for inducing an immune response in a subject, the method comprising (a) administering to the subject at least one dose of a first composition comprising a PAMP molecule; and (b) administering to the subject at least one dose of a second composition comprising a first RNA encoding at least one immunostimulatory molecule, thereby inducing an immune response.

4. The method of claim 3 wherein the immunostimulatory molecule is a single chain IL-12 (scIL-12).

5. (canceled)

6. The method of claim 4 wherein the scIL-12 comprises p35-L-p40 or p40-L-p35.

7. (canceled)

8. The method of claim 3 wherein the PAMP molecule is an innate immune receptor agonist, a glycan, a glycoconjugate, a TLR agonist, a lipoprotein, a lipopeptide, or an aqueous or oil in water emulsion formulation of glucopyranosyl lipid A (GLA).

9-15. (canceled)

16. The method of claim 3 wherein the first composition and the second composition are administered by a route independently selected from the group consisting of intratumorally, intradermally, intravenously, subcutaneously, intranodally and intramuscularly.

17-27. (canceled)

28. The method of claim 3 wherein (a) two doses; (b) three doses; (c) four doses; or (d) five doses of the first composition are administered.

29-30. (canceled)

31. The method of claim 3 wherein the RNA is self-replicating RNA (srRNA) derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

32-39. (canceled)

40. A kit comprising (a) a first composition comprising PAMP molecule; and (b) a second composition comprising a first RNA encoding at least one immunostimulatory molecule.

41. A product comprising (a) a first composition comprising a PAMP molecule; and (b) a second composition comprising a first RNA encoding at least one immunostimulatory molecule, for use in a method selected from the group consisting of (i) inhibiting tumor growth in a subject, (ii) treating cancer in a subject, and (iii) inducing an immune response in a subject.

42. The product of claim 41 wherein the immunostimulatory molecule is a single chain IL-12 (scIL-12).

43. (canceled)

44. The product of claim 42 wherein the scIL-12 comprises p35-L-p40 or p40-L-p35.

45. (canceled)

46. The product of claim 41 wherein the PAMP molecule is an innate immune receptor agonist a glycan, a glycoconjugate, a TLR agonist, a lipoprotein, a lipopeptide, or an aqueous or oil in water emulsion formulation of glucopyranosyl lipid A (GLA).

47-52. (canceled)

53. The product claim 41 wherein the first composition and the second composition are administered by a route independently selected from the group consisting of intratumorally, intradermally, intravenously, subcutaneously, intranodally and intramuscularly.

54-64. (canceled)

65. The product of claim 41 wherein (a) two doses; (b) three doses; (c) four doses; or (d) five doses of the first composition are administered.

66-67. (canceled)

68. The product of claim 41 wherein the RNA is a self-replicating RNA (srRNA) derived from an alphavirus selected from the group consisting of Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, and Buggy Creek Virus.

69. The product of claim 41 wherein the second composition comprises a cationic lipid, an ionizable lipid, a liposome, a nanoparticle, a lipid nanoparticle, a cochleate, a virosome, an immune-stimulating complex, a microparticle, a microsphere, a unilamellar vesicle, a multilamellar vesicle, an oil-in water emulsion, a water-in-oil emulsion, an emulsome, a polycationic peptide, or a cationic nanoemulsion.

70-72. (canceled)

73. The product of claim 41 wherein the second composition further comprises an adjuvant.

74. The product of claim 41 wherein the second composition comprises a second, third or fourth RNA encoding an additional immunomodulatory molecule.

75. The product of claim 41 further comprising one or more additional active agents or treatments selected from the group consisting of an immune checkpoint inhibitor, an antibody that activates a co-stimulatory pathway, a cancer chemotherapy, and radiation therapy.

76-82. (canceled)

83. The product of claim 75, wherein the immune checkpoint inhibitors is selected from the group consisting of: remelimumab, anti-OX40, PD-L1 monoclonal antibody, pembrolizumab, nivolumab, CT-011, BY55 monoclonal antibody, AMP224, BMS-936559, MPLDL3280A, MSB0010718C, and Yervoy/ipilimumab.

84-87. (canceled)
